SEQUENCE LISTING (16 sequences)

SEQ ID NO: 1 - DNA, 20 bp, Artificial Sequence
  Synthetic oligonucleotide
aacaacatgccagtgatgga

SEQ ID NO: 2 - DNA, 20 bp, Artificial Sequence
  Synthetic oligonucleotide
taccaggtttttggctcacc

SEQ ID NO: 3 - DNA, 18 bp, Artificial Sequence
  Synthetic oligonucleotide
agtgcagtggcgcgatct

SEQ ID NO: 4 - DNA, 24 bp, Artificial Sequence
  Synthetic oligonucleotide
ggcagaagaattgctggaatctag

SEQ ID NO: 5 - DNA, 22 bp, Artificial Sequence
  Synthetic oligonucleotide
ctctctgctcctcctgttcgac

SEQ ID NO: 6 - DNA, 19 bp, Artificial Sequence
  Synthetic oligonucleotide
tgagcgatgtggctcggct

SEQ ID NO: 7 - DNA, 24 bp, Artificial Sequence
  Synthetic oligonucleotide
acttcttcagggcaggaactctga

SEQ ID NO: 8 - DNA, 24 bp, Artificial Sequence
  Synthetic oligonucleotide
tacaactcggacttgctgttgctc

SEQ ID NO: 9 - DNA, 29 bp, Artificial Sequence
  Synthetic oligonucleotide
agtatcgattcaataaaagttggctcaac

SEQ ID NO: 10 - DNA, 27 bp, Artificial Sequence
  Synthetic oligonucleotide
atatctagaaacagcaagtccgagttg

SEQ ID NO: 11 - DNA, 57 bp, Artificial Sequence
  Synthetic oligonucleotide
ccgggctcccttactttagatgatactcgagtatcatctaaagtaagggagcttttt

SEQ ID NO: 12 - DNA, 57 bp, Artificial Sequence
  Synthetic oligonucleotide
ccggcctctccaagatgtggatatactcgagtatatccacatcttggagaggttttt

SEQ ID NO: 13 - DNA, 57 bp, Artificial Sequence
  Synthetic oligonucleotide
ccggcctgaggaagaggagtatcttctcgagaagatactcctcttcctcaggttttt

SEQ ID NO: 14 - DNA, 57 bp, Artificial Sequence
  Synthetic oligonucleotide
ccggcgagatggaattaacagtcttctcgagaagactgttaattccatctcgttttt

SEQ ID NO: 15 - DNA, 57 bp, Artificial Sequence
  Synthetic oligonucleotide
ccggcccaccaatttggaaggcattctcgagaatgccttccaaattggtgggttttt

SEQ ID NO: 16 - protein, 11 aa, Artificial sequence
  Synthetic peptide
YGRKKRRQRRR

(19) United States
(12) Patent Application Publication
(10) Pub. No.: US 2012/0164148 A1
(43) Pub. Date: Jun. 28, 2012

(54) Compositions Containing JARID1B Inhibitors and Methods for Treating Cancer

(75) Inventors: Alexander O. Roesch, Homburg-Beeden (DE); Meenhard Herlyn, Wynnewood, PA (US)

(73) Assignee: The Wistar Institute, Philadelphia, PA (US)

(21) Appl. No.: 13/388,583

(22) PCT Filed: Aug. 6, 2010
(86) PCT No.: PCT/US10/44643

§ 371 Date: Mar. 5, 2012

Related U.S. Application Data
(60) Provisional application No. 61/329,782, filed on Apr. 30, 2010.
(60) Provisional application No. 61/232,069, filed on Aug. 7, 2009.

(51) Int. Cl. A61K 31/713; A61K 31/7105; C12N 5/02; A61K 39/395; A61P 35/00; A61P 35/04; A61K 31/135; A61K 33/24

(52) U.S. Cl. 424/142.1; 514/647; 514/44 A; 514/44 R; 424/649; 424/133.1; 435/375

(57) ABSTRACT

The present invention features compositions and methods for treating cancers such as melanoma, which have a subpopulation of self-renewing JARIDIB-positive cells essential to maintenance and metastatic progression of the cancer.

### INTRODUCTION

This application claims benefit of priority to U.S. Provisional Application Ser. Nos. 61/232,069, filed Aug. 7, 2009, and 61/329,782 filed Apr. 30, 2010, the contents of which are incorporated herein by reference in their entireties.

This invention was made with government support under contract number CA25874 awarded by the National Institutes of Health. The government has certain rights in the invention.

### BACKGROUND OF THE INVENTION

Malignant melanoma is an aggressive tumor of neuroectodermal origin that can be cured if excised in an early stage, however, once disseminated to distant organs, the median survival of melanoma patients drops below nine months (Gogas, et al. (2007) Cancer 109:455-464). For decades, melanomas have been known for their intratumoral heterogeneity regarding histo-morphological, genetic, epigenetic, and functional criteria both in vivo and in vitro (Albino, et al. (1981) J. Exp. Med. 154:1764-1778; Houghton, et al. (1987) J. Exp. Med. 165:812-829; Kath, et al. (1991) Cancer Res. 51:2205-22111 Rastetter, et al. (2007) Histol. Histopathol. 22:1005-1015; Helmbold, et al. (2005) J. Am. Acad. Dermatol. 52:803-809; Barranco, et al. (1994) Cancer Res. 54:5351-5356; Lotem, et al. (2003) Cancer Genet. Cytogenet. 142:87-91; Morita, et al. (1998) J. Invest. Dermatol. 111:919-924; Nakayama, et al. (2001) Am. J. Pathol. 158:1371-1378). This enormous diversity paired with the potential for continuous tumor self-renewal previously led to the question of whether melanomas follow the cancer stem cell model with a melanoma stem cell on top of a tumor differentiation pyramid (Reya, et al. (2001) Nature 414:105-111; Zabierowski & Herlyn (2008) J. Clin. Oncol. 26:2890-2894). Since the initial validation of the cancer stem cell model for acute myeloid leukemia (Bonnet & Dick (1997) Nat. Med. 3:730-737), cancer stem cells have also been identified in solid tumors, such as breast (Wright, et al. (2008) Breast Cancer Res. 10:R10), colon (Ricci-Vitiani, et al. (2007) Nature 445:111-115), prostate (Vander Griend, et al. (2008) Cancer Res. 68:9703-9711), pancreas (Li, et al. (2007) Cancer Res. 67:1030-1037), and brain cancer (Singh, et al. (2003) Cancer Res. 63:5821-5828), mainly based on the expression of surface markers. It has been reported that the B cell marker CD20 is indicative for increased self-renewal capacity of melanoma sphere cells after propagation in human embryonic stem cell medium (Fang, et al. (2005) Cancer Res. 65:9328-9337). In addition, CD133 (Monzani, et al. (2007) Eur. J. Cancer 43:935-946), ABCB1 (Keshet, et al. (2008) Biochem. Biophys. Res. Commun. 368:930-936), ABCB5 (Schatton, et al. (2008) Nature 451:345-349), and ABCG2 (Monzani, et al. (2007) Eur. J. Cancer 43:935-946) have been used to characterize stem-like subpopulations in melanomas with frequencies broadly ranging between ˜0.0001% and 0.1% of the total population depending on the marker and experimental method used. However, it has been pointed out that modifications to xenotransplantation assays, which currently represent the standard assay to assess tumor self-renewal (Clarke, et al. (2006) Cancer Res. 66:9339-9344), can dramatically increase the frequency of tumor-initiating/melanoma stem cells up to 25% of unsorted cells, i.e., independent from any supposed stem cell marker (Quintana, et al. (2008) Nature 456:593-598). Besides the conclusion that basically every melanoma cell might initiate a tumor if the host system is susceptible enough, this finding suggested the existence of ‘melanoma stem cells’ (Clarke, et al. (2006) Cancer Res. 66:9339-9344; Adams & Strasser (2008) Cancer Res. 68:4018-4021). Melanomas may not be hierarchically organized into different subpopulations of tumorigenic and non-tumorigenic cells and the cancer stem cell model might not account for melanoma heterogeneity. Therefore there is a need to determine whether, within an established tumor microenvironment, continuous tumor maintenance is similarly assured by each individual melanoma cell or if distinct subpopulations are more suited as a resource for replenishment. In the latter scenario, the potential to continuously maintain tumors might be independent of the capacity to initiate new tumors in host organisms and might not follow the ‘static’ hierarchical cancer stem cell model, particularly when the enormous plasticity and heterogeneity of melanomas are taken into consideration.

### SUMMARY OF THE INVENTION

The present invention features a composition composed of a cancer therapeutic agent and a JARID1B inhibitor. In certain embodiments, the cancer therapeutic agent is a radiotherapeutic agent such as brachytherapy, or a chemotherapeutic agent such as an alkylating agent, antimetabolite, anthracycline, vinca alkaloid, taxane, topoisomerase inhibitor, monoclonal antibody or kinase inhibitor. In specific embodiments, the alkylating agent is cisplatin, carboplatin, oxaliplatin, mechlorethamine, cyclophosphamide, or chlorambucil; the antimetabolite is azathioprine or mercaptopurine; the vinca alkaloid is Vincristine, Vinblastine, Vinorelbine or Vindesine; the taxane is paclitaxel; the topoisomerase inhibitor is amsacrine, irinotecan, topotecan, etoposide, etoposide phosphate or teniposide; the monoclonal antibody is Trastuzumab, Cetuximab, Rituximab, Ipilimumab, Tremelimumab or Bevacizumab; and the kinase inhibitor is imatinib mesylate, sorafenib, Raf265 (CHIR-265), PLX4032, PD0325901, or AZD6244. In some embodiments, the JARID1B inhibitor inhibits the activity of JARID1B and is a histone H3 lysine demethylase inhibitor such as tranylcypromine. In other embodiments, the JARID1B inhibitor inhibits the expression of JARID1B and is an antisense, ribozyme, or RNAi molecule. In specific embodiments, the JARID1B inhibitor is an RNAi molecule of SEQ ID NO:11, SEQ ID NO:12, or SEQ ID NO:13. A pharmaceutical composition containing the cancer therapeutic agent and JARID1B inhibitor in admixture with a pharmaceutically acceptable carrier is also provided.

The present invention also embraces methods for decreasing self-renewal of tumor cells and inhibiting metastatic progression of a cancer using a JARID1B inhibitor. In accordance with some embodiments of these methods, the JARID1B inhibitor inhibits the activity of JARID1B and is a histone H3 lysine 4 demethylase inhibitor such as tranylcypromine. In accordance with other embodiments, the JARID1B inhibitor inhibits the expression of JARID1B and is an antisense, ribozyme, or RNAi molecule. In specific embodiments, the JARID1B inhibitor is RNAi molecule of SEQ ID NO:11, SEQ ID NO:12, or SEQ ID NO:13. In certain embodiments, the cancer or tumor is an epithelial cancer or tumor, e.g., breast cancer, prostate cancer, esophageal cancer, adenocarcinoma, squamous cell carcinoma or melanoma. In accordance with the method for inhibiting metastatic progression of a cancer, some embodiments further include the use of a cancer therapeutic agent such as a chemotherapeutic agent; a radiotherapeutic agent; an immune modulator; surgery; a molecule-targeted drug; immune therapy including vaccination, lymphocytes, or dendritic cells; or a combination thereof. A pharmaceutical composition containing a JARID1B inhibitor and formulated for transdermal or topical administration is also provided.

The invention also features a method for treating cancer by administering to a subject in need of treatment an effective amount of a cancer therapeutic agent in combination with an agent that modulates JARID1B. In certain embodiments, the cancer therapeutic agent is a chemotherapeutic agent such as an alkylating agent, antimetabolite, anthracycline, vinca alkaloid, taxane, topoisomerase inhibitor, monoclonal antibody or kinase inhibitor; a radiotherapeutic agent such as external beam radiotherapy, external beam teletherapy, brachytherapy, sealed source radiotherapy, systemic radioisotope therapy or unsealed source radiotherapy; an immune modulator; surgery; a molecule-targeted drug; immune therapy including vaccination, lymphocytes, or dendritic cells; or a combination thereof. In specific embodiments, the alkylating agent is cisplatin, carboplatin, oxaliplatin, mechlorethamine, cyclophosphamide, or chlorambucil; the antimetabolite is azathioprine or mercaptopurine; the vinca alkaloid is Vincristine, Vinblastine, Vinorelbine or Vindesine; the taxane is paclitaxel; the topoisomerase inhibitor is amsacrine, irinotecan, topotecan, etoposide, etoposide phosphate or teniposide; the monoclonal antibody is Trastuzumab, Cetuximab, Rituximab, Ipilimumab, Tremelimumab or Bevacizumab; and the kinase inhibitor is imatinib mesylate, sorafenib, Raf265 (CHIR-265), PLX4032, PD0325901, or AZD6244. In one embodiment, the agent that modulates JARID1B is a JARID1B inhibitor that inhibits the activity or expression of JARID1B. JARID1B inhibitors that inhibit activity include histone H3 lysine 4 demethylase inhibitors such as tranylcypromine, whereas JARID1B inhibitors that inhibit JARID1B expression include antisense, ribozyme, or RNAi molecules. In particular embodiments, the JARID1B inhibitor is an RNAi molecule of SEQ ID NO:11, SEQ ID NO:12, or SEQ ID NO:13. In another embodiment, the agent that modulates JARID1B is a JARID1B activator that increases the activity or expression of JARID1B. In particular embodiments, the JARID1B activator is a membrane-transducable JARID1B fusion protein. In other embodiments, the cancer is an epithelial cancer such as breast cancer, prostate cancer, esophageal cancer, adenocarcinoma, squamous cell carcinoma or melanoma.

### BRIEF DESCRIPTION OF THE DRAWINGS

FIG. 1 shows that melanomas contain a subpopulation of slowly-proliferating cells characterized by increased JARID1B expression. FIG. 1A, Flow cytometric isolation of side population cells was performed. Semiquantitative RT-PCR screening of side population cells (SP; Hoechst 33342 low) from WM3734 and from WM35 melanoma cells showed a significant upregulation of JARID1B compared to the main populations (MP; Hoechst 33342 high) (*p<0.05, t-test). FIG. 1B, Cells retaining maximum label (LR) displayed significantly enhanced JARID1B expression compared to non-label-retaining (nLR) cells in semiquantitative RT-PCR (*p<0.05, t-test).

FIG. 2 shows the in vitro self-renewal capacity of the JARID1B-positive subpopulation. FIG. 2A, While there was no significant difference in proliferation between J/EGFP-positive and J/EGFP-negative cells within the first 4 days after sorting (MTS assay), after day 10, the progeny of J/EGFP-positive cells started to proliferate significantly faster (p<0.05, ANOVA). FIG. 2B, Clonogenic assays confirmed the enhanced growth capacity of single J/EGFP-positive cells. Sorted cells had been seeded at a clonal density (5000 cells per 6 well) and were grown for days (*p<0.01, t-test). FIGS. 2C and 2D, J/EGFP-positive cells had both a higher potential to form three dimensional colonies in 0.35% hESCM4-soft agar (FIG. 2C) (*p<0.001, t-test) 21 days after sorting and to self-renew again into heterogeneous melanoma spheres in limited dilution assays (FIG. 2D) (*p=0.013, Fisher's exact test) 30 days after sorting. Shown is one representative from at least two independent experiments.

FIG. 3 shows that single xenografted melanoma cells initiate tumors regardless of JARID1B expression. Xenotransplantation growth curves after subcutaneous injection of 100 (FIG. 3A), 10 (FIG. 3B) or 1 WM3734 melanoma cell (FIG. 3C) into NOD/LtSscidIL2Rγ^{null} mice were used to show initiation of tumor growth independent of the J/EGFP expression status. Unsorted cells were used as a control. Growth curves were stopped when the first mouse of the respective series had to be sacrificed due to high tumor load (>1000 mm³). The remaining mice were further observed.

FIG. 4 shows sh JARID1B knockdown in WM3734, WM35, and WM3899 melanoma cells. FIG. 4A, After puromycin selection of positively transduced WM3734, WM3899, and WM35 cells, manual cell counts confirmed the initial increase of proliferation followed by growth flattening when compared to respective sh scrambled controls. Knockdown and cell counts were done in three independent consecutive approaches and are summarized (p<0.05 for all cell lines, ANOVA). All data are displayed as relative fold growth normalized to the cell number on day 1. FIG. 4B, Limited (single cell) dilution assays determined the reduction of melanoma sphere formation in JARID1B knockdown cells compared to controls (*p<0.05, Fisher's exact test). FIG. 4C, After embedding cells at clonal density (5000 cells per 6 well) into 0.35% Tu2%-soft agar, JARID1B knockdown led to a significant reduction in 3D colony formation (*p<0.0001 and **p<0.01, t-test). Depicted are representative results from at least three independent experiments.

FIG. 5 shows In vivo exhaustion after knockdown of JARID1B. FIG. 5A, In vivo tumor growth was exhausted after 4 passages of serial xenotransplantation of JARID1B knockdown cells (WM3734) in NOD/LtSscidIL2Rγ^{null} mice (n=5 per sample) compared to controls. 10⁴ cells were injected per passage. Tumor growth was measured weekly and was terminated when the first tumor of the series reached 1000 mm³. FIG. 5B, When the in vivo proliferation capacity of JARID1B knockdown cells was displayed as normalized growth ratio (tumor volumes of sh JARID1B tumors divided by volumes of sh scrambled tumors), loss of continuous tumor growth became clearly visible over the cumulative growth phase of 27 weeks. FIG. 5C, Five weeks after subcutaneous injection of 5×10⁵ WM3899 melanoma cells into NOD/LtSscidIL2Rγ^{null} mice (n=5 per sample), a significant decrease of spontaneous metastasis into lungs after JARID1B knockdown was found. Shown is one representative from two independent experiments (p<0.05, t-test). H&E whole lung sections were used to count the number of macro- and micrometastases at 20× magnification. Two representative sections per lung were analyzed. Counts were normalized to 100 mm³ of lung sections using Image Pro Plus software.

FIG. 6 shows that JARID1B+ cells survive targeted therapy. Left panels, Relative enrichment of the J/EGFP+subpopulation after 72 hours of treatment with a BRAF inhibitor (PLX4720), bortezomib (VEL), temozolomide (TMZ), or salinomycin (SAL) as determined by flow cytometry. The threshold (dotted line) for the J/EGFP+ subpopulation was set at 5% (DMSO control) based on JARID1B expression studies. Depicted box plots represent three independently performed experiments with flow cytometric determination of J/EGFP expression beyond the indicated threshold. Right panels, The number of viable cells within the total population was decreasing during drug treatment.

FIG. 7 shows the increased in vivo susceptibility of melanoma to conventional anti-cancer therapy as a result of stable depletion of the JARID1B-expressing subpopulation. Xenotransplanted WM3734 melanomas (mean tumor volume ˜200 mm³) were treated with 20 μg bortezomib (dissolved in PBS and injected intraperitoneally) on days 20, 22, 25, and 27 after xenotransplantation. Compared to the control (squares, sh scrambled), JARID1B knocked-down tumors (“x”) showed a significantly lower tumor volume with p=0.024 (MANOVA).

### DETAILED DESCRIPTION OF THE INVENTION

Mounting evidence has suggested that quiescent cancer stem cells play important roles in tumor self-renewal and resistance to therapy of various cancers. However, in advanced melanomas, which are notoriously resistant to all available therapies, the concept of a static stem cell hierarchy with only a minute tumor-initiating subpopulation has not been previously confirmed. Using the H3K4 demethylase JARID1B (KDM5B/PLU-1/RBP2-H1) as a biomarker, a small subpopulation of slowly-proliferating melanoma cells has been identified that cycles with doubling times >4 weeks within the rapidly proliferating main population. Isolated JARID1B-positive melanoma cells give rise to highly proliferative progeny and show high self-renewal capacity. Knock-down of JARID1B leads to initial growth acceleration followed by exhaustion, which indicates that the JARID1B-positive subpopulation is essential for continuous tumor maintenance. Expression of JARID1B is dynamically regulated and does not follow a hierarchical cancer stem cell model because JARID1B-negative cells can become positive and even single melanoma cells irrespective of selection are tumorigenic. Thus, targeting this subpopulation of JARID1B-positive cells, which are required for continuous tumor maintenance, but whose phenotype is plastic, represents an effective means to exhaust tumor growth and development. Since current anti-cancer strategies predominately affect the rapidly proliferating tumor bulk, the slowly-proliferating self-renewing JARID1B-positive cells represent a novel therapeutic target. Accordingly, the present invention embraces compositions and methods for treating cancer, which target the larger population of JARID1B-negative cells as well as the subpopulation of slow-cycling, self-renewing JARID1B-positive cells.

For the purposes of the present invention, a JARID1B-negative cell is used in the context of cancer to refer to those tumor cells that exhibit no or undetectable JARID1B expression and have a high proliferative capacity as compared to normal, non-cancerous cells and JARID1B-positive cells, e.g., as determined by growth rates or expression of proliferative markers such as Ki-67. According to the invention, it is these rapidly proliferating JARID1B-negative tumor cells that are targeted by conventional cancer therapeutic agents.

In contrast, a JARID1B-positive cell is used in the context of cancer to refer to those cells which exhibit an elevated level of JARID1B expression as compared to normal cells or tumor bulk cells, exhibit a slow-cycling phenotype (e.g., having a doubling time of >4 weeks), and/or are capable of self-renewal. For the purposes of the present invention, the term “self-renewing” or “self-renewal” of a JARID1B-positive cell refers to the ability of the cell to resemble the parental tumor heterogeneity either in vitro, e.g., heterogeneous microarchitecture of spheres, or in vivo, e.g., heterogeneity of xenografted melanoma (cell morphology, pigmentation, vascularization).

To target both JARID1B-negative cells and self-renewing JARID1B-positive cells, the present invention features compositions composed of one or more cancer therapeutic agents and one or more agents that inhibit JARID1B. A cancer therapeutic is used in the conventional sense to refer to chemotherapeutic and radiotherapeutic agents that control or kill malignant or cancer cells. As used herein, cancer chemotherapeutic agents refer to cytotoxic agents that induce apoptosis and/or impair mitosis of rapidly dividing cells. Cancer chemotherapeutic agents of use in accordance with the present invention include, but are not limited to, those exemplified herein as well as any suitable alkylating agent (e.g., cisplatin, carboplatin, oxaliplatin, mechlorethamine, cyclophosphamide, and chlorambucil); antimetabolite (e.g., azathioprine and mercaptopurine); anthracycline; plant product including vinca alkaloids (e.g., Vincristine, Vinblastine, Vinorelbine, and Vindesine) and taxanes (e.g., paclitaxel); and topoisomerase inhibitor (e.g., amsacrine, irinotecan, topotecan, etoposide, etoposide phosphate, and teniposide), which affect cell division or DNA synthesis and/or function; as well as monoclonal antibodies (e.g., Trastuzumab, Cetuximab, Rituximab, Ipilimumab, Tremelimumab and Bevacizumab) and protein kinase inhibitors (e.g., imatinib mesylate, sorafenib, Raf265 (CHIR-265), PLX4032, PD0325901, and AZD6244), which directly target protein kinases that have been oncogenically activated in human cancers such as colorectal cancer, lung cancer, pancreatic cancer, an melanoma (see Dancey & Sausville (2003) Nature Rev. Drug Discover. 2:296-313; Roberts & Der (2007) Oncogene 26:3291-3310).

A radiotherapeutic agent refers to an agent the produces ionizing radiation that damages cellular DNA. Radiotherapy is conventionally provided as external beam radiotherapy (EBRT or XBRT) or teletherapy, brachytherapy or sealed source radiotherapy, and systemic radioisotope therapy or unsealed source radiotherapy. The differences relate to the position of the radiation source; external is outside the body, brachytherapy uses sealed radioactive sources placed precisely in the area under treatment, and systemic radioisotopes are given by infusion or oral ingestion. In this regard, when used in the context of a composition of the present invention, a radiotherapeutic is intended to main a radioactive agent used in brachytherapy. When used in the context of the methods of the present invention, a cancer therapeutic includes all forms of radiotherapy routinely used in the art.

The selection of one or more appropriate cancer therapeutics for use in the composition and methods of the invention can be carried out by the skilled practitioner based upon various factors including the condition of the patient, the mode of administration, and the type of cancer being treated.

A JARID1B inhibitor is intended to include agents that inhibit the activity or expression of JARID1B. Such inhibition can be indirect, or direct by binding to JARID1B protein or nucleic acids. JARID1B (Jumonji:AT rich interactive domain 1B (RBP2-like), also known as Putative DNA/Chromatin-Binding Motif 1 (PUT1, PLU-1), Lysine-Specific Demethylase 5B (KDM5B), Retinoblastoma-Binding Protein 2, homolog 1 (RBBP2H1, RBP2-H1), and Retinoblastoma-Binding Protein 2, homolog 1A (RBBP2H1A) is known in the art as a histone H3 lysine 4 demethylase. See, e.g., Lu, et al. (1999) J. Biol. Chem. 274:15633-45; Vogt, et al. (1999) Lab. Invest. 79:1615-27; Kashuba, et al. (2000) Europ. J. Hum. Genet. 8:407-413; Christensen, et al. (2007) Cell 128:1063-76; Yamane, et al. (2007) Mol. Cell. 6:801-12. Accordingly, histone 3 lysine 4 demethylase activity (shown for LSD1/KDM1A) can be inhibited using histone H3 lysine 4 demethylase inhibitors such as tranylcypromine (Lee (2006) Chem. Biol. 13:563-567), or agents identified in screening assays for JARID1B-specific inhibitors. Such screening assays typically involve contacting JARID1B, or a cell expressing the same, with a test agent and determining whether the test agent inhibits the demethylase activity of JARID1B or a cellular phenotype associated with JARID1B activity. Compounds which can be screened in accordance with such a method can derived from libraries of agents or compounds. Such libraries can contain either collections of pure agents or collections of agent mixtures. Examples of pure agents include, but are not limited to, proteins, polypeptides, peptides, antibodies, nucleic acids, oligonucleotides, carbohydrates, lipids, synthetic or semi-synthetic chemicals, and purified natural products.

JARID1B expression can be inhibited using, e.g., antisense, ribozyme, or RNAi molecules or techniques known in the art. In particular embodiments, RNA interference or RNAi is employed. This technique involves introducing into a cell double-stranded RNA (dsRNA), having a sequence corresponding to the exonic portion of the target gene. The dsRNA causes a rapid destruction of the target gene's mRNA. See, e.g., Hammond, et al. (2001) Nature Rev. Gen. 2:110-119; Sharp (2001) Genes Dev. 15:485-490. Procedures for using RNAi technology are described by, for example, Waterhouse, et al. (1998) Proc. Natl. Acad. Sci. USA 95(23):13959-13964. Typically, siRNAs are about 20 to 23 nucleotides in length. The target sequence that binds the siRNA can be selected experimentally or empirically. For example, empirical observations have indicated that 51RNA oligonucleotides targeting the transcriptional start site of the target gene (Hannon (2002) Nature 418:244-51) or targeting the 3′ untranslated region of the mRNA (He and Hannon (2004) Nature 5:522-531) are more effective at blocking gene expression. Further, siRNA target sites in a gene of interest are selected by identifying an AA dinucleotide sequence, typically in the coding region, and not near the start codon (within 75 bases) as these may be richer in regulatory protein binding sites which can interfere with binding of the 51RNA (see, e.g., Elbashir, et al. (2001) Nature 411: 494-498). The subsequent 19-27 nucleotides 3′ of the AA dinucleotide can be included in the target site and generally have a G/C content of 30-50%.

RNAi can be performed, for example, using chemically-synthesized RNA. Alternatively, as disclosed herein, suitable expression vectors are used to transcribe such RNA either in vitro or in vivo. In vitro transcription of sense and antisense strands (encoded by sequences present on the same vector or on separate vectors) can be effected using, for example, T7 RNA polymerase, in which case the vector can contain a suitable coding sequence operably-linked to a T7 promoter. The in vitro-transcribed RNA can, in certain embodiments, be processed (e.g., using RNase III) in vitro to a size conducive to RNAi. The sense and antisense transcripts are combined to form an RNA duplex which is introduced into a target cell of interest. Other vectors can be used, which express small hairpin RNAs (shRNAs) which can be processed into shRNA-like molecules. Various vector-based methods are described in, for example, Brummelkamp, et al. (2002) Science 296(5567):550-3; Lee, et al. (2002) Nat. Biotechnol. 20(5):500-5; Miyagashi and Taira (2002) Nat. Biotechnol. 20(5):497-500; Paddison, et al. (2002) Proc. Natl. Acad. Sci. USA 99(3):1443-8; Paul, et al. (2002); and Sui, et al. (2002) Proc. Natl. Acad. Sci. USA 99 (8):5515-20. According to particular embodiments of the present invention, the shRNA molecule is expressed using a lentivirus-based expression system. Such lentivirus systems are known in the art and available from sources such as Dharmacon (Lafayette, Colo.) and Sigma. Kits for production of dsRNA for use in RNAi are also available commercially, e.g., from New England Biolabs, Inc. and Ambion Inc. (Austin, Tex., USA). Methods of transfection of dsRNA or plasmids engineered to make dsRNA are routine in the art. Exemplary shRNA molecules targeting the coding region and 3′UTR of JARID1B are listed in Table 3.

In an alternative embodiment, the inhibition of JARID1B can be achieved using an indirect approach such as an immune therapy-based approach, which includes vaccination with JARID1B protein or nucleic acids, or via modulation of JARID1B-affected or -affecting pathways such as notch and HIF signaling, or gene therapy.

According to this invention, the cancer therapeutic and JARID1B inhibitor can be provided as a composition prepared as a combination of formulations (e.g., the composition includes or comprises a formulation containing a cancer therapeutic agent, and a formulation containing a JARID1B inhibitor), or the composition can be prepared as a single unitary formulation (e.g., the composition includes or comprises a formulation containing a cancer therapeutic agent and a JARID1B inhibitor). Moreover, when the composition is prepared as individual or a combination of formulations, said formulations can be the same, e.g., all tablets; or different, e.g., a capsule formulation and a liquid formulation. In addition, when taken as individual formulations, said formulations can be taken simultaneously or consecutively, e.g., within hours or days of each other.

For therapeutic use, the therapeutic agent and/or JARID1B inhibitor of the invention is desirably formulated as a pharmaceutical composition or medicament for use in cancer treatment. Such formulations contain the active ingredient(s) in admixture with a pharmaceutically acceptable carrier. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable carrier or vehicle, such as a liquid or solid filler, diluent, excipient, or solvent encapsulating material, is involved in carrying or transporting the active ingredient from one organ, or portion of the body, to another organ, or portion of the body. Each carrier must be acceptable in the sense of being compatible with the other ingredients of the formulation and not injurious to the subject being treated.

Examples of materials that can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The compositions of the present invention can be administered parenterally (for example, by intravenous, intraperitoneal, subcutaneous or intramuscular injection), topically (including buccal and sublingual), orally, intranasally, intravaginally, rectally, intratumorally or transdermally depending upon the formulation and/or cancer to be treated.

The selected dosage level will depend upon a variety of factors including the activity of the particular active agent employed, the route of administration, the time of administration, the rate of excretion or metabolism of the particular agent being employed, the duration of the treatment, other drugs, compounds and/or materials used in combination with the particular agent employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of an agent at levels lower than that required in order to achieve the desired therapeutic effect and gradually increase the dosage until the desired effect is achieved. This is considered to be within the skill of the artisan and one can review the existing literature on a specific compound or similar compounds to determine optimal dosing.

As an alternative to JARID1B inhibition, other embodiments of this invention embrace compositions (both single agent and combination compositions) containing agents that increase expression and/or activation of JARID1B. Such an increase in JARID1B expression is expected to induce a slowly-proliferating state in the bulk of tumor cells such that the whole of the tumor becomes quiescent and the patients has an increased survival time. Alternatively, increased JARID1B expression may induce the tumor cells to undergo apoptosis. Increases in JARID1B in the treatment of cancer can include, e.g., the use of membrane-transducable JARID1B fusion proteins wherein JARID1B is fused to a known protein transduction domain such as PTD-4, HIV TAT, PTD-3, PTD-5, PTD-6, PTD-7, ANTₚ, or Transportin (Ho, et al. (2001) Cancer Res. 61:474-477; Schwartz and Zhang (2000) Curr. Opin. Moi. Ther. 2:2). As demonstrated herein, JARID1B is required for continuous maintenance of tumor growth and metastatic progression. Thus, the present invention embraces methods for decreasing long-term self-renewal of tumor cells, treating cancer and inhibiting metastatic progression of a cancer by modulating the expression or activity of JARID1B alone, or in combination with a cancer therapeutic agent. Such combination therapy can be carried out consecutively or simultaneously.

According to this invention, a method for decreasing long-term self-renewal of tumor cells involves contacting a tumor with an agent that modulates JARID1B so that long-term (e.g., 12, 14, 16, 18, 20 or more weeks) self-renewal of the tumor cells is decreased or inhibited as compared to tumor cells which have not been contacted with the JARID1B inhibitor. In one embodiment, the JARID1B modulator activates JARID1B, i.e., a JARID1B activator. In another embodiment, the JARID1B modulator inhibits JARID1B i.e., a JARID1B inhibitor. In so far as JARID1B modulation reduces the subpopulation of JARID1B-positive cells, cancer treatment and inhibition of metastatic progression is facilitated. Effectiveness of JARID1B modulation for decreasing self-renewal can be determined using the methods exemplified herein or any other suitable method known in the art. Desirably, the agent selectively inhibits self-renewal to the extent that a 70%, 80%, 90%, 95% or 99% level of cell death is achieved. Tumor cells which can be treated in accordance with this method of the invention include, but are not limited to, tumors wherein JARID1B expression is elevated throughout cells of the tumor or expressed in a subpopulation of cells. Such tumors include epithelial tumors such as breast tumor, prostate tumor, esophageal tumor, adenocarcinoma, squamous cell carcinoma and melanoma. In particular embodiments, the tumor is a melanoma tumor. In some embodiments, the tumor cell is contacted in vitro. In other embodiments, the tumor cell is contacted in vivo.

As indicated, JARID1B is required for tumor maintenance such that treatment of cancer solely with a conventional therapeutic agent can be insufficient to fully treat the cancer. Thus, the present invention embraces a method for treating cancer by administering to a subject in need of treatment an effective amount of a cancer therapeutic (e.g., conventional cytostatic or cytotoxic agents and immune modulators; radiation therapy; surgery; molecule-targeted drugs; or any kind of immune therapy including vaccination, lymphocytes, dendritic cells; or a combination thereof) in combination with an agent that inhibits JARID1B. By way of illustration, a subject with cancer can be treated by surgery to remove the bulk of the tumor and subsequently treated with a conventional cytostatic agent and agent that inhibits JARID1B. As used herein, treatment of cancer encompasses either reducing the growth of a tumor in the subject, reducing the clinical symptoms associated with tumor growth in the subject, and/or increasing survival time as compared to a subject not receiving treatment. For the purposes of the present invention, “treatment” refers to both therapeutic treatment and prophylactic measures (e.g., in cancer recurrence). As such, those in need of treatment include those already with the cancer as well as those who have been treated for cancer and are at risk of recurrence. Subjects who can be treated in accordance with the present invention include mammals, such as humans, domestic and farm animals, and zoo, sports, or pet animals, e.g., dogs, horses, cats, cows, etc. Preferably, the mammal herein is human. Cancers which can be treated in accordance with this method of the invention include, but are not limited to, cancers wherein JARID1B expression is elevated throughout cells of the tumor or expressed in a subpopulation of cells. Such cancers include epithelial cancers such as breast cancer, prostate cancer, esophageal cancer, squamous cell carcinoma, adenocarcinoma, and melanoma. In particular embodiments, the cancer is a melanoma.

In so far as JARID1B is associated with a subpopulation of slow-cycling, self-renewing cells, which are not targeted by conventional anti-cancer approaches, it is further contemplated that in addition to combination therapies composed of 1) a drug that targets rapidly proliferating cells and 2) a drug that kills slowly-proliferating cells, combination therapies composed of 1) a drug that targets rapidly proliferating cells and 2) a drug that transforms slowly-proliferating cells into rapidly proliferating cells, which can then be targeted by the first drug, are also of use. In either case, the drugs can be administered simultaneously or consecutively.

A method for inhibiting metastatic progression of a cancer is also embraced by the present invention. According to this embodiment, a subject in need of treatment is administered an effective amount of an agent that inhibits JARID1B so that metastatic progression of the subject's cancer is inhibited. Subjects benefiting from such treatment include those diagnosed with a cancer known to metastasize or move from the site of initiation to other tissues or organs. Such cancers include epithelial cancers such as breast cancer, prostate cancer, esophageal cancer, squamous cell carcinoma, adenocarcinoma, and melanoma. In particular embodiments, the cancer is a melanoma.

The invention is described in greater detail by the following non-limiting examples.

### Example 1

### Experimental Procedures

Melanoma Samples and Cell Culture. Fresh human melanoma tissues and cells were obtained according to standard procedures, isolated and maintained in 2% fetal bovine serum (FBS)-substituted melanoma medium (‘Tu2%’) Smalley, et al. (2005) Am. J. Pathol. 166:1541-1554; Satyamoorthy, et al. (1997) Melanoma Res. 7 Suppl 2:S35-42). According to the isolation of neural stem cells and cancer stem cells from brain tumors by culturing of neurospheres (Yuan, et al. (2004) Oncogene 23:9392-9400; Galli, et al. (2004) Cancer Res. 64:7011-7021), melanoma spheres were propagated in mouse embryonic fibroblast (MEF)-conditioned human embryonic stem cell medium (hESCM) according to conventional methods (Fang, et al. (2005) supra). Before use, MEF-conditioned hESCM was mixed with fresh hESCM medium at a 7:3 ratio (‘hESCM4’) and basic fibroblast growth factor was added at 4 ng/ml. Depending on the cell line, sphere formation could be observed 2-6 weeks after starting culture in hESCM4. Spheres were dissociated by collagenase I/IV (Sigma, St. Louis, Mo.) and mechanical treatment, i.e., 200-250 units per ml DMEM (Cellgro) at 37° C. for 10 minutes plus subsequent pipetting. Cell viability was assured microscopically and/or by 7-AAD dead cell exclusion. The consistency of cellular genotypes and identities was confirmed by DNA fingerprinting using a Coriell microsatellite kit (Coriell, Camden, N.J.). The lentiviral vector constructs for stable knockdown of JARID1B and the scrambled control were purchased from Sigma. Lentiviral pLU-CMV-pBlast and pLU-CMV-EGFP vectors were used to clone pLU-JARID1 Bprom-EGFP-Blast and pLU-CMV-EGFP-Blast. The JARID1B main promoter was PCR-cloned from human genomic DNA (Promega, Madison, Wis.) and verified by DNA sequencing based on the published sequence. Lentiviral infections were performed according to conventional methods (Smalley, et al. (2005) supra). Selection of positive clones was carried out by treatment with puromycin (knockdown vectors) or blasticidin (promoter vectors).

In Vitro Self-Renewal Assays. Continuous survival of cells in a stem cell microenvironment (hESCM4) was measured after 5×10⁴ cells had been incubated in T25 culture flasks for 4-7 weeks depending on the cell line. During this time, cells either detached and formed melanoma spheres, stayed attached, or died. Fresh medium was added weekly. The live/dead cell ratio was assessed using trypan blue and a hematocytometer after dissociation of spheres. Quantification of melanoma sphere formation (sphere self-renewal) was done by limited (single cell) dilution assays. Briefly, cells were seeded at a ratio of 0.5 cell per well in 96-well plates to avoid doublets. Using an OLYMPUS CKX41SF phase contrast microscope, wells containing one cell were marked after 2 hours. Development of spheres was assessed after 20-30 days. To exclude delayed growth within the remaining wells, plates were periodically re-assessed for another 3 weeks.

Xenotransplantation Assays and Metastasis Model. To address continuous tumor maintenance in vivo, serial xenotransplantation assays with JARID1B knockdown vs. control cells were performed in NOD/LtSscidIL2Rγ^{null} mice. For every passage, 10⁴ cells in MATRIGEL®/Tu2% at a 1:1 dilution were injected subcutaneously in mice (5 mice per sample). One passage included: injection, tumor growth, tumor dissection, cell isolation (mechanical and collagenase I/IV treatment) and purification. Melanoma cell purification was done by three-day-puromycin treatment confirmed by MCAM flow cytometry. Verification of knockdown was carried out by QRT-PCR. Titration xenotransplantation of J/EGFP-sorted cells was done for 100, 10 and 1 cell per injection (5 mice per sample, 4 injections per mouse). One hundred and 10 cell dilutions were based on FACS counts and were verified microscopically. Preparation of single cell injections was performed using standard methods (Quintana, et al. (2008) supra). Tumor growth was measured weekly using a caliper and was terminated when the first tumor of the series reached 1000 mm³. Metastatic progression was measured in a spontaneous metastasis model. WM3899 melanoma cells (5×10⁵) were injected subcutaneously into NOD/LtSscidIL2Rγ^{null} mice (5 mice per sample) and mice were incubated for five weeks. Formalin-fixed, paraffin-embedded (FFPE) sections of whole lungs were H&E-stained and the numbers of macro- and micrometastases were microscopically determined (20× magnification). Two representative frontal sections per lung were analyzed. Counts were normalized to 100 mm³ of lung sections using Image Pro Plus software.

In Vitro and In Vivo Label Retaining (LR) Assays. In vitro LR of cells was done using the PKH26 Red Fluorescent Cell Linker Kit for general membrane labeling (Sigma). Initially, the dye/cell/volume conditions were optimized. One hundred percent labeling efficiency with low toxicity was reached when 10⁶ dissociated sphere cells were incubated with 1 μM PKH26 in a reaction volume of 100 μl. Before labeling, dead sphere cells were isolated by 7-Amino-Actinomycin D (7-AAD) fluorescence-activated cell sorting (FACS). Flow cytometry and fluorescence microscopy verified initial PKH26-LR efficiency and, after 4 weeks of cell proliferation in hESCM4, was used to detect the PKH26-LR subpopulation. Dead cells were again excluded with 7-AAD after respective compensation. In vivo BrdU-LR was performed according to standard methods (Kiel, et al. (2007) Nature 449:238-242; Molofsky, et al. (2006) Nature 443:448-452). Briefly, 10,000 WM3734 cells that had been infectedwith pLU-JARID1 Bprom-EGFP-Blast were xenotransplanted into NOD/LtSscidIL2Rγ^{null} mice (n=10). Five of the mice were given a single intraperitoneal injection of 1.5 mg BrdU in Dulbecco's Phosphate Buffered Saline (DPBS) and were subsequently maintained on 1 mg/ml BrdU in the drinking water for 12 days. Five mice served as controls. Tumors were grown for an additional six weeks and were processed as described herein. A BD PHARMINGEN APC BrdU Flo Kit was used for detection.

Immunoassays. Cultured cells were prepared by pelleting and embedding the cells into Sakura's Tissue Tek O.C.T. compound for cryopreservation; and either separating the cells by FACS and cytospinning the cells onto glass slides; or growing the cells in 6-well or 24-well plates. Cryosections were fixed in acetone and blocked in phosphate buffered saline (PBS) containing 1% bovine serum albumin (BSA). Cytospun and in vitro grown cells were fixed in 4% paraformaldehyde (PFA) in PBS and were permeabilized with 0.1% TRITON X-100 before being blocked in PBS containing normal goat serum at 1:100 or 1% BSA. Primary antibodies used were monoclonal mouse anti-Ki-67 (prediluted; Zymed, South San Francisco, Calif.) and polyclonal rabbit anti-human JARID1B (Roesch, et al. (2005) Mod. Pathol. 18:1249-1257) diluted to 5-50 μg/ml. Secondary antibodies used for immunofluorescence microscopy were goat anti-mouse ALEXA FLUOR 568, goat anti-rabbit ALEXA FLUOR 488, and goat anti-rabbit ALEXA FLUOR 568 (all from Invitrogen, Carlsbad, Calif.).

To histochemically detect JARID1B immunoreactivity in cryosections, goat anti-rabbit biotinylated IgG (1:200; Vector Labs, Burlingame, Calif.) and the ABC Elite Kit (Vector Labs), with AEC (3 amino-9 ethylcarbazole) as a final substrate, were used. FFPE material was processed according to known methods (Roesch, et al. (2005) supra). Samples were evaluated with NIKON TE2000 inverted and NIKON E600 upright microscopes.

For immunoblotting of whole cell lysates, equal amounts of proteins were solubilized (30-150 μg) in NUPAGE LDS sample buffer (Invitrogen). Samples were separated on NUPAGE 4-12% Bis-Tris Gels (Invitrogen) and were electrophoretically transferred onto polyvinylidene difluoride membranes (Millipore). Primary antibodies were incubated at 4° C. overnight in Tris-buffered saline containing 0.1% TWEEN-20 (TBST) and 5% milk (TEST-milk). Primary antibodies used were polyclonal rabbit anti-JARID1B directed to residues 784-883 (1:1000; Strategic Diagnostics, Newark, Del.) and monoclonal mouse anti-human (3-actin (1:500; Sigma). After washing with TBST and a 1-hour incubation with either anti-rabbit or anti-mouse horseradish peroxidase-conjugated secondary antibody diluted 1:5000 in TBST-milk (Amersham), immunocomplexes were visualized using the enhanced chemoluminiscence system (Amersham). After analysis, western blots were stripped once and reprobed to demonstrate equal protein loading. For quantification, signals were densitometrically normalized to β-actin or PDI with ImageJ 1.38× software (NIH).

Semiquantitative Real Time Reverse Transcriptase (RT)-Polymerase Chain Reaction (PCR). Total RNAs from samples and from standard human reference RNA (Stratagene, La Jolla, Calif.) were reverse-transcribed using the SUPERSCRIPT First-Strand cDNA synthesis kit (Invitrogen). Power SYBR Green PCR Master Mix (Applied Biosystems, Foster City, Calif.) was used with 100 ng cDNA template and 70 nM primers for the evaluation of target gene and GAPDH expression. A negative control without cDNA was run with each assay. Amplifications were performed in an ABI PRISM 7000 Sequence Detection System (Applied Biosystems). Thermal cycler conditions were 95° C. for 10 minutes, then 40 cycles of 15 seconds at 95° C. followed by 1 minute at 60° C. All experiments were performed at least in triplicate. Baseline and threshold values for genes were set using the ABI 7000 PRISM Software. mRNA expression was calculated using the relative standard curve method according to Applied Biosystems' Chemistry Guide. Expression ratios of controls were normalized to 1. Primers were either designed, acquired through literature searches, or from the Harvard primer bank. The primers employed are listed in Table 1.

TABLE 1

| | | SEQ |
|---|---|---|
| Primer | Sequence (5′->3′) | ID NO: |
| JARID1B_forward_1 | aacaacatgccagtgatgga | 1 |
| JARID1B_reverse_1 | taccaggtttttggctcacc | 2 |
| JARID1B_forward_2 | agtgcagtggcgcgatct | 3 |
| JARID1B_reverse_2 | ggcagaagaattgctggaatctag | 4 |
| GAPDH_forward | ctctctgctcctcctgttcgac | 5 |
| GAPDH_reverse | tgagcgatgtggctcggct | 6 |

JARID1B primer pairs 1 and 2 targeted different regions of the coding sequence of JARID1B cDNA. The primer sets were used to confirm each other and to exclude differential expression of the JARID1B splicing variants PLU-1 and RBP2-H1 (Roesch, et al. (2005) supra; Barrett, et al. (2002) Int. J. Cancer 101:581-588; Vogt, et al. (1999) Lab Invest. 79:1615-1627; Lu, et al. (1999) J. Biol. Chem. 274:15633-15645; Wilsker, et al. (2005) Genomics 86:242-251).

PCR-Cloning of the Human JARID1B Promoter. Based on published sequence information (Catteau, et al. (2004) Int. J. Oncol. 25:5-16), a nested PCR reaction using the primers listed in Table 2 was established to clone the human JARID1B main promoter from human genomic DNA (100 ng; Promega). Using various 5′ and 3′ deletion constructs of an initially 6.64 kb-spanning genomic fragment (which was expected to harbor the JARID1B promoter), the activity of this promoter region in different cell types has been shown (Catteau, et al. (2004) supra). Other regions of the constructs were subcloned from pLU-CMV-pBlast and pLU-CMV-EGFP.

TABLE 2

| | | SEQ |
|---|---|---|
| Primer | Sequence (5′->3′) | ID NO: |
| JARID1B_5′ UTR forward | acttcttcagggcaggaactctga | 7 |
| JARID1B_3′ UTR reverse | tacaactcggacttgctgttgctc | 8 |
| JARID1B_prom forward | agtatcgattcaataaaagttggctcaac¹ | 9 |
| JARID1B_prom reverse | atatctagaaacagcaagtccgagttg² | 10 |

¹Contains a ClaI site.
²Contains a XbaI site.

As a lentiviral control construct, pLU-CMV-EGFP-Blast with CMV promoter-driven enhanced green fluorescent protein (EGFP) expression was cloned. Stably infected WM3734 melanoma cells were cultured in conventional medium and were sorted by FACS for EGFP-positive and EGFP-negative cells (maximum and minimum thresholds were set at 5%). Semiquantitative RT-PCR proved that CMV-driven EGFP expression was not associated with differences in expression of endogenous JARID1B.

Cell Proliferation Assays. To assess cellular proliferation, cells were seeded in triplicate in 6-well plates (10⁴ cells per well) and were counted each day using a hematocytometer and trypan blue staining to exclude dead cells. Colorimetric proliferation assays were performed in 96-well plates as 8-fold measurements. For the [3-(4,5-dimethylthiazol-2-yl)-5-(3-carboxymethoxyphenyl)-2-(4-sulfophenyl)-2H-tetrazolium (MTS) assay (Promega), time courses of 500-2000 cells per well in 96-well plates were performed depending on the cell line. For BrdU incorporation assays (Roche Applied Science, Indianapolis, Ind.), 5000 cells per well in 96-well plates were seeded overnight. BrdU signals were normalized to MTS signals to compensate for possible inaccuracy of seeded cell numbers. Both assays were performed according to the manufacturers' recommendations.

Clonogenic and Colony Formation Assays. To measure clonal growth of single cells in 2D culture, 5000 cells were seeded per well in 6 well plates (clonal density). After 21 days in hESCM4 medium, clones that had grown were digitally quantified using Image Pro software. Three dimensional colony formation was assessed after 5000 cells had been embedded into soft agar in 6-well plates (end concentration 0.35% agar in PBS/medium 1:1) and grown over 14-21 days depending on the cell line. Anchorage-dependent growth was inhibited by a bottom layer of 1% soft agar. Tu2% or hESCM4 culture medium was put on top and was changed biweekly. Colony numbers were assessed microscopically and confirmed digitally by Image Pro software. All assays were performed in triplicate.

Flow Cytometry and Fluorescence Activated Cell Sorting. For detection of JARID1B promoter-driven EGFP signals, adherent cells were harvested with 0.05% trypsin and spheres were dissociated as described herein. Dead cells were excluded by staining for 7-AAD. After sorting, aliquots were microscopically checked and were cultured for a short-time to exclude disproportional enrichment of debris or apoptotic cells. Fluorescence-activated cell sorting was carried out using a CYTOMATION MOFLO cytometer (DakoCytomation). Flow cytometric detection of surface markers was done according to standard procedures. Briefly, cells were fixed in 4% paraformaldehyde (PFA) in PBS for 10 minutes and then were blocked with 1% BSA in PBS for 10 minutes at room temperature. Primary and secondary antibodies were incubated for 10 minutes at room temperature. Before and after antibody incubations, cells were washed three times with 1% BSA in PBS. Samples were analyzed using an EPICS XL instrument (Beckman-Coulter). Monoclonal antibodies were follows: anti-CD20 (APC-conjugated; BD PHARMINGEN), anti-CD133 (PE; Miltenyi Biotech), anti-p75/NGFR (APC; Miltenyi Biotech), and anti-MCAM (FITC; R&D Systems). Isotype-matched mouse APC- (BD PHARMINGEN), PE- (R&D Systems), or FITC-conjugated (R&D Systems) antibodies were used as controls. Flow cytometric isolation of side population cells was performed according to established methods (Goodell, et al. (1996) J. Exp. Med. 183:1797-1806).

ShRNA Analysis. The shRNA clones used in the shRNA knockdown of JARID1B were obtained from Sigma. The sequence and target location are listed in Table 3.

TABLE 3

| | | | SEQ |
|---|---|---|---|
| | Target | | ID |
| shRNA | Location | Sequence | NO: |
| JARID1B_59 | CDS | CCGGGCTCCCTTACTTTAGATGAT | 11 |
| | | ACTCGAGTATCATCTAAAGTAAGG | |
| | | GAGCTTTTT | |
| JARID1B_60 | CDS | CCGGCCTCTCCAAGATGTGGATAT | 12 |
| | | ACTCGAGTATATCCACATCTTGGA | |
| | | GAGGTTTTT | |
| JARID1B_61 | CDS | CCGGCCTGAGGAAGAGGAGTATCT | 13 |
| | | TCTCGAGAAGATACTCCTCTTCCT | |
| | | CAGGTTTTT | |
| JARID1B_62 | CDS | CCGGCGAGATGGAATTAACAGTCT | 14 |
| | | TCTCGAGAAGACTGTTAATTCCAT | |
| | | CTCGTTTTT | |
| JARID1B_58 | 3′UTR | CCGGCCCACCAATTTGGAAGGCAT | 15 |
| | | TCTCGAGAATGCCTTCCAAATTGG | |
| | | TGGGTTTTT | |

CDS, coding sequence.
UTR, untranslated region.
Underlined sequences indicate siRNA sequences.

Spontaneous Metastasis Model. Metastatic progression was measured in a spontaneous metastasis model. WM3899 melanoma cells (5×10⁵) were injected subcutaneously into NOD/LtSscidIL2Rγ^{null} mice (5 mice per sample) and incubated for five weeks. FFPE sections of whole lungs were H&E-stained and the number of macro- and micrometastases were microscopically determined (20× magnification). Two representative frontal sections per lung were analyzed. Counts were normalized to 100 mm³ of lung sections using IMAGE PRO PLUS software.

Statistics. To determine the statistical significance of growth curves, ANOVA for repeated measures was applied and confirmed by Student's t-test. Differences in sphere formation capacities (limited dilution assays) were statistically determined using Fisher's Exact Test. For all other experiments, the Student's t-test was used. A p-value of less than 0.05 was considered significant. As software tools, SAS version 9.2 using Proc Freq and MICROSOFT EXCEL were used.

### Example 2

### JARID1B as a Marker for Slowly-Proliferating Melanoma Cells

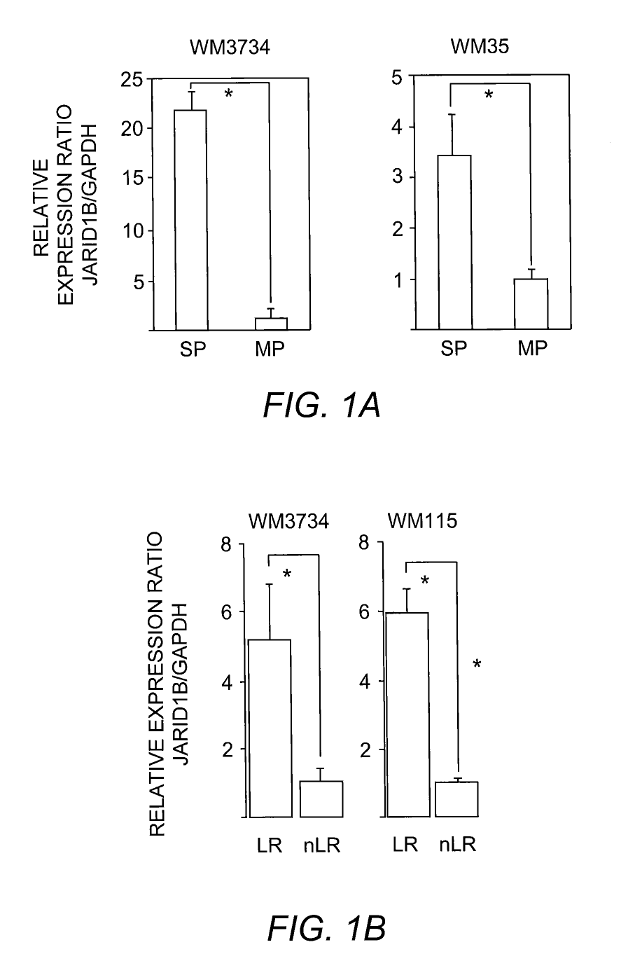

Following the concept that tumor cells with stemness properties are quiescent and show increased Hoechst 33342 efflux potential, side population analysis (Monzani, et al. (2007) supra; Hadnagy, et al. (2006) Exp. Cell Res. 12:3701-3710; Goodell, et al. (1996) J. Exp. Med. 183:1797-1806; Grichnik, et al. (2006) J. Invest. Dermatol. 126:142-153; Frank, et al. (2005) Cancer Res. 65:4320-4333) was applied to enrich for G0/1 phase cells. In subsequent genome-wide expression profiling of side population cells from different melanoma cell lines compared to their respective main populations, the transcriptional regulator and chromatin remodeling factor JARID1B was identified as a candidate marker for slowly-proliferating side population cells. Further, semiquantitative RT-PCR confirmed the statistical significance of the upregulation of JARID1B in side population melanoma cells (FIG. 1A). JARID1B (KDM5B/PLU-1/RBP2-H1/RBBP2H1a; Lu, et al. (1999) J. Biol. Chem. 274:15633-15645; Vogt, et al. (1999) Lab Invest. 79:1615-1627; Roesch, et al. (2005) Mod. Pathol. 18:1249-1257; Kashuba, et al. (2000) Eur. J. Hum. Genet. 8:407-413) is a member of the highly conserved family of jumonji/ARID1 (JARID1) histone 3 K4 demethylases which are involved in tissue development, cancer, and normal stem cell biology (Yamane, et al. (2007) Mol. Cell. 25:801-812 Klose, et al. (2007) Cell 128:889-900; Wilsker, et al. (2005) Genomics 86:242-251; Secombe & Eisenman (2007) Cell Cycle 6:1324-1328; Christensen, et al. (2007) Cell 128:1063-1076; Iwase, et al. (2007) Cell 128:1077-1088). Demethylation of H3K4 by JARID1B has been reported to play a role in the cell fate decision of embryonic stem cells by blockage of terminal differentiation (Dey, et al. (2008) Mol. Cell. Biol. 28:5312-5317). In cancer cells, JARID1B functions as a transcriptional regulator of oncogenes, e.g., BRCA1 in breast cancer, via direct interaction with respective promoter sites (Scibetta, et al. (2007) Mol. Cell. Biol. 27:7220-7235; Tan, et al. (2003) J. Biol. Chem. 278:20507-20513). Depending on the cancer context, JARID1B is associated with either positive (melanoma) or negative (breast cancer) cell cycle control (Yamane, et al. (2007) supra; Scibetta, et al. (2007) supra; Roesch, et al. (2006) J. Invest. Dermatol. 126:1850-1859; Roesch, et al. (2008) Int. J. Cancer 122:1047-1057).

Furthermore, retention of the membrane dye PKH26 was used as a marker for melanoma cells with low cell doubling and proliferation rates. Dissociated 7-AAD-negative WM3734 sphere cells, cultured in stem cell medium, were incubated with PKH26 at a concentration sufficient to label 100% of cells. The sphere model was chosen because stem cell medium not only better separates the JARID1B-positive subpopulation from the bulk, but also forms a more distinct label-retaining subpopulation. As the bulk of cells divided during the following 4-week period, the dye was diluted into subsequent daughter cells. The doubling time of unsorted WM3734 cells is approx. 48 hours. Only a small percentage of cells (2%) retained the maximum amount of label, indicating that those cells had not divided within the 4 weeks. A cell line-specific artifact was excluded by replication of these findings in a second melanoma cell line with a different biological phenotype, WM115. PKH26 label-retaining cells (LR cells) of both WM3734 and WM115 expressed JARID1B at significantly higher levels than bulk cells in semi-quantitative RT-PCR. JARID1B upregulation in label-retaining cells was statistically significant for both cell lines analyzed, WM3734 and WM115 (p<0.05, t-test, FIG. 1B).

Following the observation that slowly-cycling tumor cells often show increased Hoechst 33342 efflux potential (Addla, et al. (2008) Am. J. Physiol. Renal Physiol. 295: F680-F687; Goodell, et al. (1996) J. Exp. Med. 183:1797-1806; Grichnik, et al. (2006) J. Invest. Dermatol. 126:142-153; Hadnagy, et al. (2006) Exp. Cell Res. 312:3701-3710; Ho, et al. (2007) Cancer Res. 67:4827-4833), side population analysis of two melanoma sphere lines was conducted. Subsequent RT-PCR confirmed the significant upregulation of JARID1B also in side population cells. Genome-wide expression profiling of label-retaining and side population cells from a panel of four melanoma cell lines indicated that other sternness-related jumonji family members, e.g., JMJD1A (Loh, et al. (2007) Genes Dev. 21:2545-2557), might also be differentially regulated. However, except for JARID1B, which was upregulated both in LR and in side population cells across different cell lines, no consistent expression pattern was detected by RT-PCR and microarrays for other jumonji genes. Taken together, these data indicate that even within highly proliferative melanomas, a JARID1B-positive subpopulation resides in a slowly-proliferating state and melanoma heterogeneity might also apply to the rate of cell proliferation.

### Example 3

### Melanomas Contain Scattered Cells with Increased JARID1B Expression and Slowly-Proliferating Phenotype

In normal adult tissues, JARID1B is marginally expressed with dramatic peak expression levels in regenerative tissues like the testis and bone marrow (Vogt, et al. (1999) supra; Roesch, et al. (2005) supra; Barrett, et al. (2002) Int. J. Cancer 101:581-588). Since JARID1B was also found to be upregulated in breast cancer and its knock-down led to a decrease of tumor growth, it was initially referred as a testis-cancer antigen (Lu, et al. (1999) supra; Yamane, et al. (2007) supra; Barrett, et al. (2002) supra). In neuroectodermal melanocytic tumors, the expression patterns of JARID1B are different. JARID1B is highly expressed in benign melanocytic nevi (moles) which typically are characterized by oncogene-induced senescence (Michaloglou, et al. (2005) Nature 436:720-724). However, in aggressive malignant melanomas and in proliferating melanoma metastases, there are only scattered cells present with considerably high JARID1B expression, indicating a subpopulation with a unique biology. JARID1B immunostaining of a large series of melanoma patient samples has been conducted (Roesch, et al. (2005) supra) and scattered, highly positive cells with predominantly nuclear and minor cytoplasmic staining were observed amidst the bulk of negative cells.

When the immunohistochemical analysis was expanded to cultured melanoma cells, very similar staining patterns were observed. Sphere formation from different tissues has been proposed to be a common growth characteristic of self-renewing cells, including neural crest-derived stem cells (Weiss, et al. (1996) Trends Neurosci. 19:387-393; Toma, et al. (2005) Stem Cells 23:727-737; Dontu, et al. (2003) Genes Dev. 17:1253-1270; Singh, et al. (2004) Nature 432:396-401). Using immunohistochemistry, a total panel of six established melanoma cell lines (WM3734, WM35, WM3899, WM115, WM3523, and WM3854) with diverse phenotypes and genotypes (derived from RGP, VGP or metastatic melanomas; harboring BRAFV600E, BRAFV600D, BRAFG464E, PTEN, c-kit, or p53 mutations) were analyzed under different growth conditions. Compared to conventional Tu2% medium, which showed broad “intra-culture” heterogeneity of JARID1B expression levels that were independent of the melanoma cell line analyzed, growth in hESCM4 medium elicited a more distinct signal heterogeneity, i.e., small-sized highly JARID1B-positive cells (frequency approx. 5-10%) surrounded by the JARID1B-negative bulk population. Using digital quantitation of pseudocolored immunosections, an average of 4.8% JARID1B-positive cells was confirmed across randomly selected sphere sections (10 representative images out of 5 different melanoma cell lines). This pattern was similar to that observed in patients' tumor samples, indicating that growth under stem cell conditions recapitulates the phenotype observed in vivo. Notably, JARID1B-positive cells mostly lacked expression of the proliferation marker Ki-67 in both cultured cells and a series of patient tumors (Table 4), indicating a correlation between a slowly-proliferating phenotype and JARID1B expression.

TABLE 4

| Patient* | Ki-67 [%]^{#} | JARID1B [%]^{#} |
|---|---|---|
| 14181/00 | 20 | 1 |
| 14180/00 | 60-70 | 1 |
| 15090/00 | 50-60 | <1 |
| 3882/00 | 70-80 | 0 |
| 1985/00 | 50-60 | 5 |
| 1336/00 | 70-80 | <1 |
| 4083/00 | 70-80 | <1 |
| 18317/00 | 50-60 | 10 |

*Sample origin was a cutaneous melanoma metastatis.
^{#}Three fields of vision were assessed with a NIKON E600 Upright Microscope (40X objective).

In so far as JARID1B has been shown to be involved in cell cycle arrest in JARID1B-transfected melanoma cells (Roesch, et al. (2006) supra), these data support the concept that even within highly proliferative melanomas, a JARID1B-positive subpopulation resides in a slowly-proliferating state. Thus, melanoma heterogeneity may also apply to the level of cell proliferation.

### Example 4

### Slowly-Proliferating Melanoma Cells Form a Distinct JARID1B-Positive Subpopulation

To analyze live cells according to different endogenous levels of JARID1B expression, a model was developed. WM3734 melanoma cells (brain metastasis, BRAFV600E) were stably infected with a lentiviral construct, which drives cytoplasmic EGFP expression controlled by the co-cloned human JARID1B main promoter (pLU-JARID1Bprom-EGFP-Blast). The WM3734 cell line (subsequently abbreviated WM3734^{JARID1Bprom-EGFP-Blast}) was selected because it exhibited two-to-three fold higher relative expression of JARID1B as compared to WM35, WM3899, and WM115 cell lines and was expected to provide better read-outs. Culturing of WM3734^{JARID1Bprom-EGFP} cells resulted in JARID1B promoter-driven EGFP expression patterns (further abbreviated as J/EGFP) that clearly reflected the anti-JARID1B immunostaining observed described herein. Flow cytometry was used to quantify the J/EGFP signals. In conventional culture, the resulting flow histogram showed a bell-shaped distribution of J/EGFP. When culture conditions were switched from conventional to stem cell medium, a biphasic distribution was seen with an unspecific first peak (same fluorescence intensity as the autofluorescence control) and a second peak representing specific J/EGFP-positive cells after ˜2-3 weeks. In mature sphere culture (>4 weeks in stem cell medium), again a bell-shaped curve was noticed although with a more prominent right shoulder was observed as compared to cells cultured in conventional medium. Using fluorescence-activated cell sorting, maximum and minimum EGFP-expressing subpopulations were isolated using thresholds based on in vitro and in vivo observations of endogenous JARID1B expression frequency (5-10% positive cells). Thus, the maximum EGFP signal (representing 5% of the total population) was scored as J/EGFP-positive, and accordingly, the minimum EGFP signal (also set at 5%) as J/EGFP-negative. Using semiquantitative RT-PCR, immunoblot analysis, and immunofluorescence microscopy, it was confirmed in both culture conditions that endogenous JARID1B levels were significantly correlated with EGFP expression. In the control construct, EGFP was driven by a co-cloned CMV promoter, which did not enrich for JARID1B-positive cells.

When flow cytometry was used to analyze stably infected WM3734 melanoma cells (WM3734^{JARID1Bprom-EGFP} cells) that had been prelabeled with PKH26 for four weeks prior, the PKH26-LR cell population displayed as a distinct, almost completely J/EGFP-positive subpopulation. Backgating showed that this population was enriched for small-sized cells, which have been reported to be typical for melanoma cells with increased self-renewal properties (Grichnik, et al. (2006) supra). Disproportional enrichment for cell debris, which can be false negative for 7-AAD, particularly in the double negative fraction, was excluded microscopically. Limited (single cell) dilution assays in stem cell medium, which requires self-renewal properties for continuous expansion as spheres (Fang, et al. (2005) supra), revealed a significantly increased sphere formation capacity of the LR-J/EGFP-double positive subpopulation after 21 days (p=0.0025, Fisher's exact test). Of note, non label-retaining, but J/EGFP-positive cells, also self-renewed into spheres, a finding which could be explained by the dynamics of the JARID1B phenotype, as shown herein. Most of the non label-retaining-J/EGFP-negative cells died within the first 3 weeks of culture in stem cell medium. However, to exclude delayed sphere formation, the limited dilution assays were periodically re-assessed for another 3 weeks, during which time no new spheres formed. To exclude possible cell culture artifacts, increased label retention of the J/EGFP-positive subpopulation was additionally confirmed in vivo using incorporated BrdU as (DNA) label. For confirmation of the label-retaining-J/EGFP-positive subpopulation in vivo, WM3734^{JARID1Bprom-EGFP} melanoma cells were xenotransplanted into NOD/LtSscidIL2Rγ^{null} mice and the developing tumor population was labeled with intraperitoneally and orally administered BrdU for 12 days (n=5). After 6 weeks of tumor proliferation and BrdU dilution into subsequent daughter cells, BrdU label-retaining-J/EGFP-positive cells were identified as a distinct subpopulation and at a similar percentage (1-2%) as seen before in vitro.

### Example 5

### The Slowly-Proliferating JARID1B-Positive Subpopulation Shows Increased In Vitro Self-Renewal

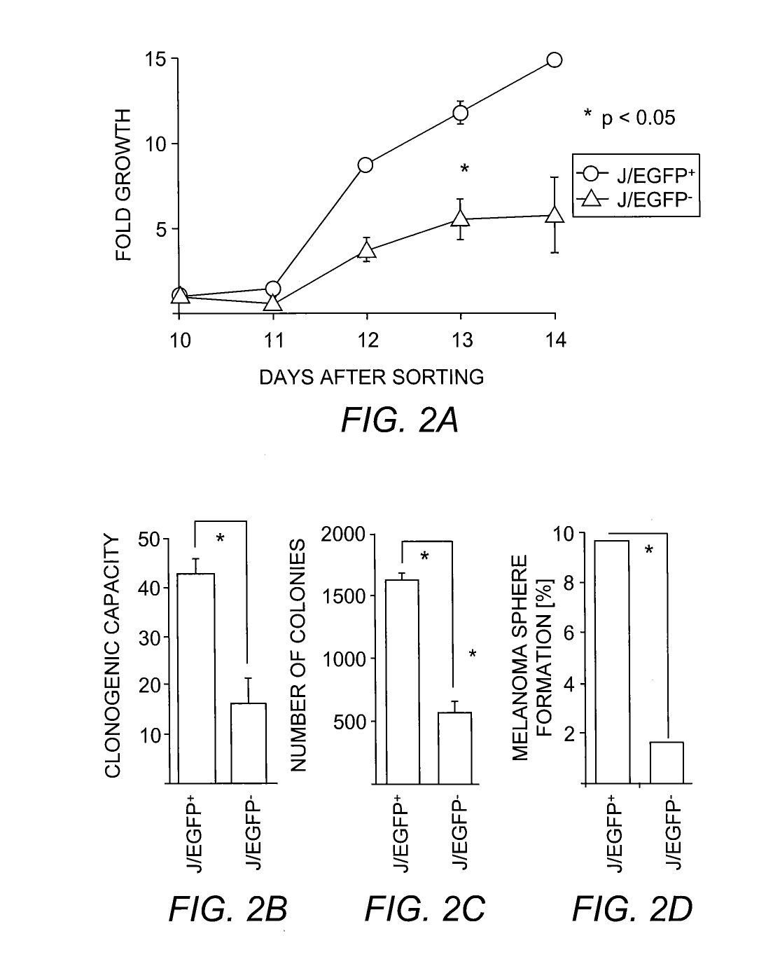

It was next determined whether isolated slowly-proliferating J/EGFP-positive cells can give rise to a progeny that resemble the parental culture heterogeneity. Melanoma spheres were used as a model because of their more distinct JARID1B expression pattern and their more heterogeneous architecture compared to adherent cells. Spheres were dissociated enzymatically and mechanically, and after removal of dead cells and debris, the single cell suspension was sorted according to J/EGFP-expression levels as described herein. Twenty-four hours after sorting, BrdU incorporation confirmed that isolated J/EGFP-positive cells were still in a slowly-proliferating state (note: slowly-proliferating cells incorporate less BrdU compared to bulk cells but once incorporated they retain it longer). Microscopic evaluation after FACS showed similar numbers of division had not significantly taken place. Cells of both isolated populations appeared healthy with no signs of disproportional enrichment for cell debris or cell death after FACS. When cell proliferation in hESCM4 medium was measured 1-4 days after sorting, no significant difference was seen between J/EGFP-positive and J/EGFP-negative cells, within the first days after sorting and reseeding in hESCM4. However, after day 10, there was a significant boost in proliferation of the J/EGFP-positive-derived progeny (p<0.05, ANOVA, FIG. 2A). Under conventional culture conditions, the difference in proliferation between the J/EGFP-positive- and -negative-derived progeny was less distinct. As confirmed by fluorescence microscopy, already seven days after sorting, J/EGFP-positive cells started to resemble the original culture heterogeneity of J/EGFP-positive and J/EGFP-negative cells. Enhanced expansion capacity of J/EGFP-positive cells after an initial delay was further confirmed by clonogenic assays in which sorted cells had been seeded at clonal density and grown for 21 days in hESCM4 medium (FIG. 2B, p<0.01, t-test). Since anchorage-independent growth is a known hallmark of cancer survival, it was determined whether J/EGFP-positive melanoma cells have different colony formation capacities compared to the tumor bulk when seeded into soft agar at clonal density. Indeed, J/EGFP-positive sphere cells formed more and larger colonies in hESCM4-soft agar than did J/EGFP-negative cells (FIG. 2C, p<0.001, t-test). It was observed that J/EGFP-negative cells formed the first visible colonies, but after 1-2 weeks, colonies derived from J/EGFP-positive cells grew dramatically faster, whereas colonies derived from J/EGFP-negative cells slowed their growth. Finally, J/EGFP-positive cells re-formed significantly more spheres in single cell dilution assays in hESCM4 medium, indicating increased self-renewal (FIG. 2D, p=0.013, Fisher's exact test). Single-seeded J/EGFP-negative cells died more often or did not form spheres during the observation period.

Since increased self-renewal of neural crest-derived cells including melanoma cells had been associated with the expression of surface markers such as CD20 (Fang, et al. (2005) surpa), CD133 (Monzani, et al. (2007) supra), and p75/NGFR (Wong, et al. (2006) J. Cell Biol. 175:1005-1015; Pietra, et al. (2009) Int. Immunol. 21:793-801), flow cytometry was used to screen WM3734 melanoma cells for double expression with J/EGFP. There was no significant correlation found in the co-expression of CD133 and p75/NGFR. In the case of CD20, a trend toward higher expression in J/EGFP-positive cells could be assumed but due to the low overall expression frequency of CD20 (0.7-2%), there was a lack of experimental consistency in replicate analyses.

Together with the expression studies in patient specimens (Roesch, et al. (2005) supra) and in melanoma cell lines, these data indicate a JARID1B-expressing subpopulation in melanomas that remains in a slowly-proliferating state, but when released from their microenvironment those cells can give rise to rapidly proliferating progeny that re-constitute the parental heterogeneity of JARID1B-positive and JARID1B-negative cells. Particularly when the culture conditions supported the survival of cells with inherent self-renewal potential, i.e., stem cell medium, single-seeded J/EGFP-positive cells were superior to single J/EGFP-negative cells regarding their potential to repopulate.

Moreover, in the case of JARID1B+ melanoma cells, stem cell medium does not simply enhance the number of positive cells, but rather the expression of JARID1B in distinct single cells, whereas the expression in surrounding cells decreases (‘focal JARID1B concentration’). Thus, as the JARID1B+ subpopulation becomes better visible in stem cell medium, the read-outs get clearer. As a consequence of this process of focal concentration, the overall JARID1B expression in the entire population can even decrease compared to regular culture conditions.

### Example 6

### The JARID1B-Positive Phenotype is not a Prerequisite for Tumor Initiation In Vivo

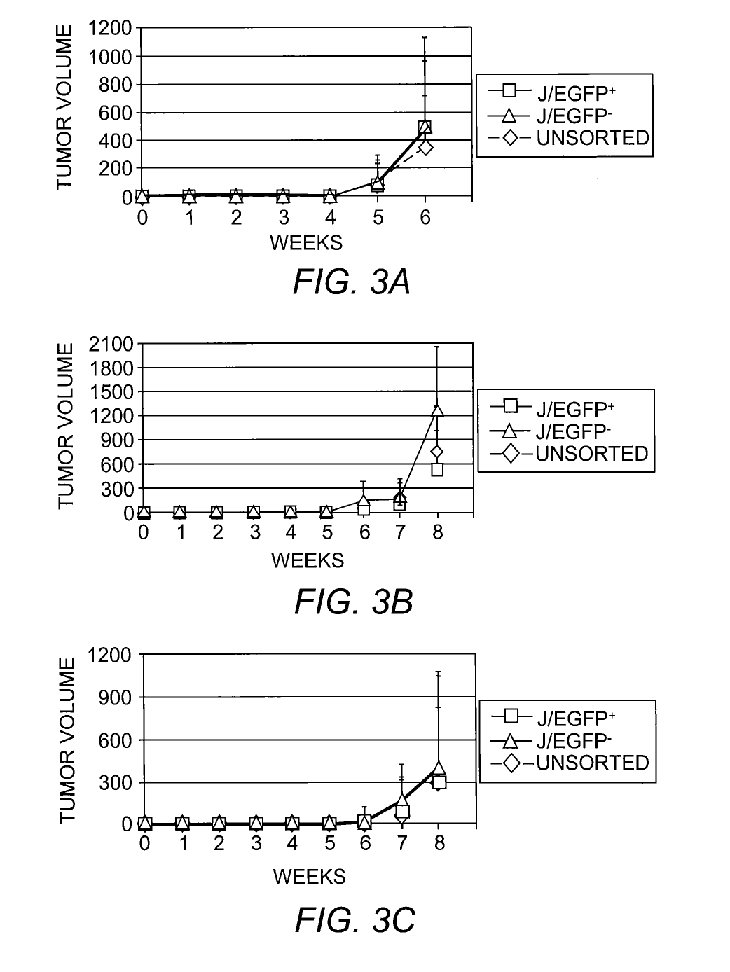

To determine the tumorigenic potentials of separated subpopulations, titrated xenotransplantation assays were performed in NOD/LtSscidIL2Rγ^{null} mice according to improved protocols (Quintana, et al. (2008) supra) (FIG. 3). To carry out this analysis, mice were subcutaneously injected with 100, 10 or 1 WM3734 cells from FACS isolated J/EGFP-positive and J/EGFP-negative subpopulations cultured in conventional medium (each n=20). Unsorted cells were injected as controls. Consistent with the observations of Quintana, et al. ((2008) supra) for a broad panel of supposed stem cell markers, the absolute tumor initiation rate of J/EGFP-positive and J/EGFP-negative cells was almost identical. The absolute ratio of tumor induction was determined 120 days after injection (Table 5).

TABLE 5

| | Cell Number per Injection | | |
|---|---|---|---|
| Cells | 100 | 10 | 1 |
| Unsorted | 18/20 | 15/20 | 9/20 |
| J/EGFP-Positive | 17/20 | 9/20 | 8/20 |
| J/EGFP-Negative | 16/20 | 12/20 | 7/20 |

In all titration steps it was observed that J/EGFP-negative cells started to grow earlier. Although not a statistically significant difference, this was of interest because it was consistent with the in vitro observations of colony formation assays (FIG. 2). A FACS-contamination of the J/EGFP-negative population with J/EGFP-positive cells as a possible bias is unlikely because induction of tumors was constantly observed in all xenografts, and also from individually injected J/EGFP-negative cells.

### Example 7

### Knockdown of JARID1B Leads to In Vitro Exhaustion of Melanoma Cells

Given the paradox of an increased in vitro self-renewal capacity without any effect on in vivo tumor initiation, it was determined whether JARID1B still could be important for the continuous maintenance of melanomas. For example, if tumor initiation on the one hand and the maintenance of established tumors on the other hand reflected two different biological processes. To address this, JARID1B was knocked down in three different established melanoma cell lines, WM3734 (brain metastasis, BRAFV600E), WM35 (RGP melanoma, BRAFV600E), and WM3899 (lung metastasis, BRAFG464E) and in primary foreskin melanocytes (FOM) as control. The efficiency of each JARID1B knockdown clone was validated at the RNA and protein levels and on a functional level using BrdU incorporation and MTS proliferation assays confirmed by manual cell counting. Out of five shRNA clones targeting different mRNA regions of JARID1B, two clones with significant knockdown phenotypes (sh JARID1B_{—}58; and JARID1B_{—}62) were selected for further experiments. Off target effects were additionally excluded by computerized shRNA sequence analysis. Unspecific effects due to knockdown or secondary regulation of other jumonji/ARID family memberswere excluded by subsequent cDNA microarrays.

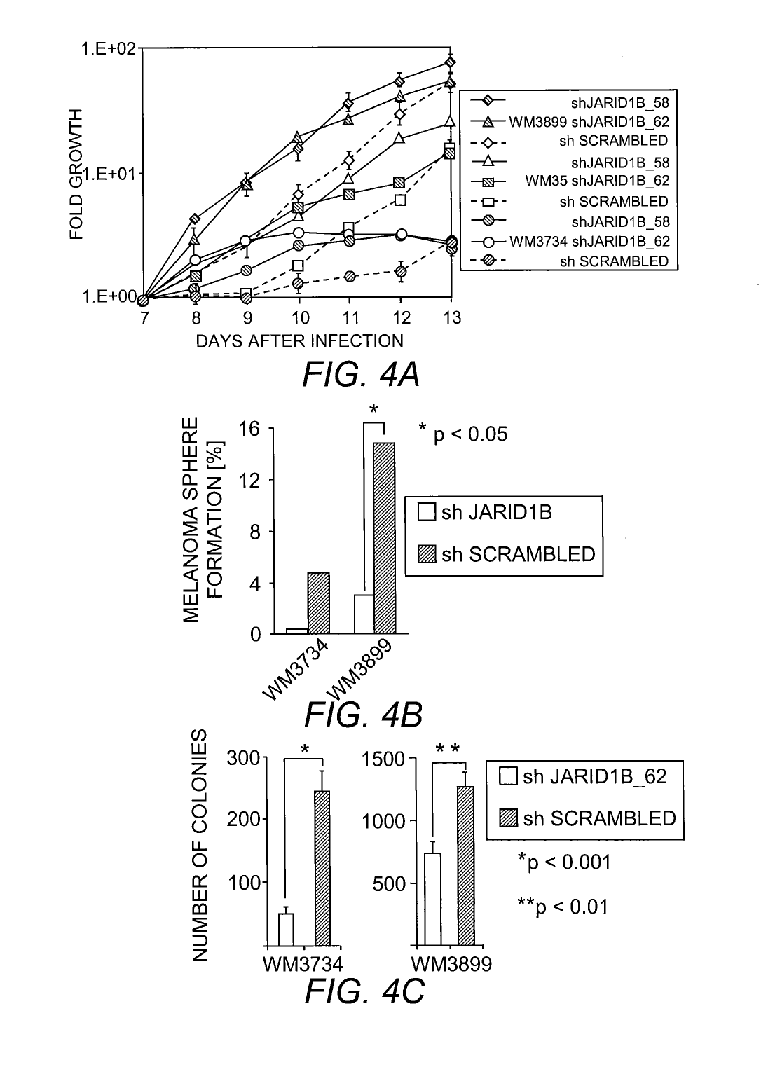

The results of this analysis indicated that JARID1B knockdown was followed by a statistically significant increase in proliferation starting from day 7 after infection (FIG. 4A, p<0.05 for all cell lines, ANOVA). However, after day 10, cell proliferation flattened and, at least in the case of WM3734 cells, even decreased although the cells were still subconfluent. This effect seemed to occur independent of the biological and genetic backgrounds of the cell lines and their respective endogenous proliferation potentials. Knockdown of different JARID1B mRNA regions showed similar results. Proliferation and pigmentation of normal melanocytes, on the other hand, remained unaffected by JARID1B knock down.

Since the melanoma cultures did not fully perish over time in conventional Tu2% medium, the culture conditions were changed to hESCM4 medium to determine whether the cells could self-renewal under stem cell conditions. While control cells formed increasing numbers of spheres following 21 days of culture in hESCM4 medium, knockdown of JARID1B led to a strong exhaustion of all three melanoma cell lines after 28 (WM3899), 37 (WM3734), and 39 (WM35) days with 86-95% cell death, indicating that in JARID1B knockdown cultures the capacity for continuous self-renewal was lost. In a confirmation experiment, primary JARID1B knocked-down WM3928MP melanoma cells also exhausted under stem cell culture conditions after 14 days. Furthermore, 30 days after seeding single cells in 96-well plates in hESCM4 medium, the decrease in sphere formation of WM3734 and WM3899 JARID1B knockdown cells compared to controls could be quantified (FIG. 4B). This was of interest because together with the observation that spheres predominantly budded from J/EGFP-positive cells, it supports the concept that JARID1B-expression defines a subpopulation which is involved in self-renewal. Consistent with the impaired colony formation properties of J/EGFP-negative cells (FIG. 2), FIG. 4C shows that both JARID1B knockdown lines formed fewer colonies in Tu2% soft agar compared to the controls after 2-3 weeks of incubation (p<0.001 and p<0.01, respectively, t-test). As seen before with J/EGFP-negative cells, knockdown cells also initially formed colonies, but further expansion decreased after several days.

### Example 8

### JARID1B is Required for the Continuous Growth of Xenografted Melanoma and for Metastatic Progression

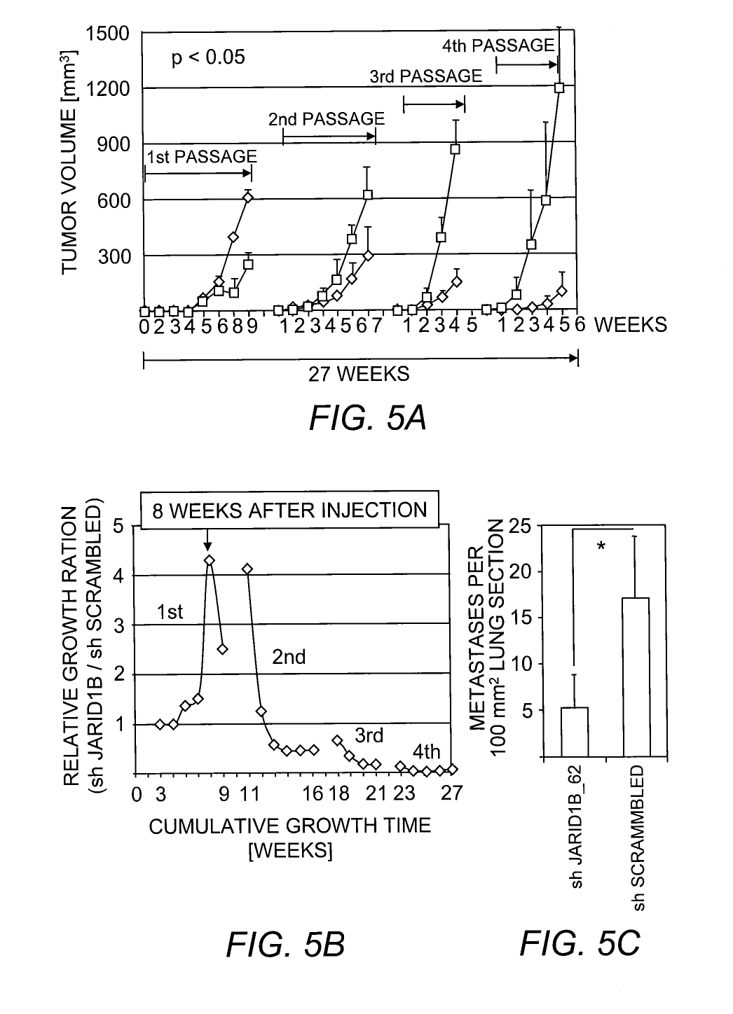

To determine whether the exhaustion phenomenon seen in vitro after JARID1B knockdown also occurred in vivo, a serial xenotransplantation assay in NOD/LtSscidIL2Rγ^{null} mice was employed. This assay allows assessment of long-term growth of implanted tumor cells without temporary restrictions due to maximum tumor size and is the only commonly accepted assay that addresses the question of continuous tumor self-renewal (Clarke, et al. (2006) supra). In the first passage of xenotransplantation, there was higher proliferation of JARID1B knockdown cells compared to the control (FIG. 5A, p<0.05, ANOVA) which mimicked the proliferation pattern seen in vitro (FIG. 4A) and the results from single injected J/EGFP-negative cells (FIG. 3). After dissociation of the tumors, melanoma cells were purified from contaminating murine cells by a short incubation with puromycin and were assessed by flow cytometry for the melanoma marker MCAM (Balint, et al. (2005) J. Clin. Invest. 115:3166-3176). The stability of JARID1B knockdown was confirmed by semiquantitative RT-PCR of an aliquot before re-injection and later by immunohistochemistry of tumor sections. Strikingly, in subsequent in vivo passages, JARID1B knockdown cells gradually lost their potential to expand (p<0.05, ANOVA). When tumor growth was displayed as relative ratio normalized to the sh scrambled control (FIG. 5B), it became apparent that over the total incubation time of 27 weeks in vivo, the proliferation of JARID1B knockdown cells peaked and then steadily exhausted as had been indicated before the in vitro experiments. Using WM3899 cells that are known to spontaneously metastasize into lungs after subcutaneous injection at the backs of NOD/LtSscidIL2Rγ^{null} mice, a significant reduction of pulmonary metastases by JARID1B knockdown cells (FIG. 5C, p<0.05, t-test) was observed, as determined by computerized quantitation of histological sections. It was concluded that the expression of JARID1B is necessary for the continuous maintenance of melanomas including systemic disease progression. This process can only be seen in long-term experiments and seems to be independent from the initial tumor formation.

### Example 9

### The JARID1B-Positive Phenotype is Dynamic

The cancer stem cell concept postulates a static hierarchy of tumor cells with a cancer stem cell at the top of a differentiation pyramid (Reya, et al. (2001) supra). Long-term culture of FACS-isolated cells indeed confirmed that the J/EGFP-positive subpopulation induces a heterogeneous daughter population composed of J/EGFP-positive and J/EGFP-negative cells as determined by immunofluorescence microscopy and flow cytometry analyses. Fourteen days after FACS and re-seeding in conventional Tu2% medium, the J/EGFP-positive-derived progeny again was composed of J/EGFP-positive cells and an increasing number of J/EGFP-negative cells. Development of a heterogeneous tumor population was also seen in vivo 56 days after J/EGFP-positive cells (100, 10 or 1) had been implanted into NOD/LtSscidIL2Rγ^{null} mice. However, after 14 days in vitro or 56 days in vivo, also J/EGFP-negative cells gave rise to a heterogenous progeny including J/EGFP-positive cells, even when derived from a single J/EGFP-negative cell. As a consequence of the re-establishing culture heterogeneity, overall JARID1B expression in the total progenies became balanced again independent of their origin. Of note, the maximum fluorescence intensity of the second generation J/EGFP-positive cells reached the fluorescence intensity of the original J/EGFP-positive cells, indicating that the phenotype is fully reversible. When sorted cells were cultured in stem cell medium rather than conventional medium, the interconversion of phenotypes was considerably decelerated. Even after 120 days of incubation, only a few J/EGFP-positive cells were found in cultures derived from J/EGFP-negative cells. Accordingly, J/EGFP-positive cells seeded in hESCM4 medium maintained a higher number of J/EGFP-positive cells. Daughter cultures from both J/EGFP-positive or J/EGFP-negative cells could be cultured for several months without any signs of exhaustion which indicated that the self renewal function of second generation J/EGFP-positive cells was fully reversible.

To determine whether interconversion also slows down when cells are kept in more diluted conditions thereby diminishing possible cross-talk, interconversion was determined at different cell confluencies. In brief, J/EGFP-negatively sorted cells were re-seeded under regular culture conditions in Tu2% (10⁵ cells per T25 flask, two flasks, #1 and #2). According to the findings presented herein, the experiment was designed for 14 days to ensure that the cells generally had enough time to revert. By day 14, flask #1 reached 95% confluency. The cells in this flask grew as very large and dense patches distributed over the entire bottom of the flask. Primarily, the centers of these patches showed high J/EGFP signals in immunofluorescence microscopy. Flask #2 was serially trypsinized during the 14-day incubation to keep the cell density stable at different (lower) confluencies. The first “daughter” flask (flask #2.1) was maintained at 10% confluency, flask #2.2 at 20-30%, flask #2.3 at 50%, and flask #2.4 at 70-80% until day 14. The results of this analysis indicated that cells between 50 and 70% confluency proliferated fastest and had to be trypsinized and diluted out most often to maintain the cell density. Cells that were maintained at clonal density (flask #2.1 and 2.2) grew very slowly and so did cells of the dense patches of flask #1. At day 14, J/EGFP levels of all flasks were assessed by flow cytometry (Table 6). To ensure specificity of J/EGFP expression, the same threshold that had been established before was applied. Thus, only cells that were beyond this threshold were considered JARID1B-positive.

TABLE 6

| | Flask | | | | |
|---|---|---|---|---|---|
| | #2.1 | #2.2 | #2.3 | #2.4 | #1 |
| Confluency | 10% | 20-30% | 50% | 70-80% | 95% |
| Cell Proliferation | Lag | Lag | Log | Log | Plateau |
| Phase | phase | phase | phase | phase | phase |
| J/EGFP-Positive | 2.4% | 2.4% | 1.2% | 0.6% | 1.8% |
| Cells* | | | | | |

*For comparison, the regular percentage of J/EGFP-positive cells in an unsorted population is 5%.

Together with the findings pertaining to JARID1B-affected JAG1/Notch signaling, in particular those which were generated in spheres with tight cell-cell contact, the results from flask #2.4 vs. #1 indicate that the JARID1B-positive phenotype is indeed dependant on cell-cell interaction (in addition to soluble factors from the culture media and the oxygen level). The interpretation of the relatively high percentage of singly reverted cells in flasks #2.1 and 2.2 is difficult since this reflects a quite rare situation in tumor biology. Tumor cells usually are in close contact to each other or to stroma. On the other hand, this finding has implications in the acquisition of sternness of single (e.g., through the blood stream metastasizing) cells.

Since conventional culture conditions seemed to allow a higher dynamics of the JARID1B-positive phenotype, clonogenic and colony formation assays were repeated for sorted cells in conventional Tu2% medium. Although J/EGFP-positive cells still showed increased colony formation, now the difference from J/EGFP-negative cells was clearly decreased compared to the hESCM4 culture conditions applied before. Next to soluble factors from the culture medium, the level of oxygen was also identified as a significant environmental factor for the dynamic regulation of JARID1B. JARID1B expression in melanoma cells rapidly enhanced under low oxygen conditions (3 days, 1% pO₂) and steadily reverted to normal expression intensity and frequency after 10 to 14 days of conventional culture at atmospheric oxygen (21% pO₂). Moreover, the level of cell-cell contact affected the determination of the JARID1B phenotype; J/EGFP-negative cells that were grown as dense patches usually developed a more distinct J/EGFP-positive subpopulation than J/EGFP-negative cells that were kept at lower density. The dynamics of the JARID1B phenotype also explains the relatively high sphere formation capacity of non-LR but J/EGFP-positive cells. These cells likely acquired the J/EGFP-associated self-renewal potential after PKH-labeling.

### Example 10

### JARID1B Affects Jagged 1/Notch 1-Signaling

It was subsequently determined whether JARID1B affects known mechanisms of self-renewal. Focus was initially placed on the bidirectional Notch signaling pathway because of its role in maintenance of neural progenitors and melanocyte stem cells (Moriyama, et al. (2006) J. Cell Biol. 173:333-339; Woo, et al. (2009) BMC Neurosci. 10:97) and its function in melanoma progression (Balint, et al. (2005) J. Clin. Invest. 115:3166-3176; Liu, et al. (2006) Cancer Res. 66:4182-4190). JARID1B transcriptionally represses the Notch ligand Jagged 1 (JAG1) through direct interaction with its promoter (Roesch, et al. (2008) supra). In a melanoma cell line that is known for its low endogenous JARID1B expression, A375-SM (Roesch, et al. (2005) supra), transient transfection with JARID1B leads to a concentration-dependent down-regulation of JAG1, whereas the results herein indicated that stable knock-down in highly JARID1B-expressing WM3734 and WM35 melanoma cells is followed by JAG1 upregulation. The JARID1B-mediated repression of JAG1 in A375-SM cells was followed by reduced cleavage of Notch 1 into its active form, Nic (Notch intracellular domain), while leaving the overall expression of Notch 1 unchanged. Non-melanocytic control cells, HEK293, were not affected by JARID1B. Since in experimentally “homogenized” cultures (by transfection or lentiviral infection), the basic character of the Notch pathway, i.e., its bidirectional signaling between cells, could be masked, naturally JARID1B-positive vs. -negative cells were used, which had been separated according to their J/EGFP-expression. Again, high JARID1B expression was associated with low JAG1, using both adherent and sphere cultures. Particularly in spheres with their clearer separation between JARID1B-positive and -negative subpopulations, high JARID1B/low JAG1 was associated with high HEY1 and HEY2 expression, which are both downstream targets for Notch signaling. The inverse expression of JARID1B, JAG1 and downstream targets amongst neighboring cells indicates that, together with JAG1/Notch 1, JARID1B is part of a complex dynamic program of sternness regulation in melanoma.

Since epithelial-mesenchymal transition (EMT) has been suggested as an alternative sternness-associated mechanism (Mani, et al. (2008) Cell 133:704-714), it was determined whether JARID1B-positive melanoma cells also show EMT or, at least, an EMT-like phenotype (because of their neuroectodermal origin, melanoma cells may not undergo classic EMT). However, none of the gene expression profiling experiments (JARID1B knockdown vs. scrambled; transient JARID1B overexpression vs. mock; and J/EGFP-positive vs. J/EGFP-negative) detected a consistent classic EMT signature in correlation with high JARID1B expression.

JARID1B transcriptionally regulates several developmental and cancer-relevant pathways (Roesch, et al. (2008) supra; Tan, et al. (2003) J. Biol. Chem. 278:20507-20513) including Notch signaling, which is involved in the maintenance of neural stem cells (Shi, et al. (2008) Crit. Rev. Oncol. Hematol. 65:43-53). Additional studies have shown that JARID1B can also be actively involved in the maintenance of the non- or slowly-proliferative state in melanoma cells via the stabilization of pRB-mediated cell cycle control (Roesch, et al. (2006) supra). Stabilization of pRB is usually understood as a tumor-suppressive mechanism because of its anti-proliferative effect, but in the long run, slow-proliferation can be also associated with tumor maintenance as the results herein indicate. Thus, JARID1B may have a dual role over time, immediately anti-proliferative but long-term tumor maintaining.

### Example 11

### JARID1B is Necessary for Maintenance of Primary Melanoma Cells

Melanomas from patients were collected and ‘direct-in vivo-cultures’ were established, i.e., the melanomas were directly maintained in NOD/SCID mice and without passage in in vitro culture. The advantage of these cell lines is that they closely resemble the original melanoma phenotype, but still provide enough cells for experiments in an ‘on-demand’ fashion. Primary cells were analyzed from an advanced human metastatic melanoma. As shown for established WM3734, WM35, and WM3899 melanoma cell lines, the primary melanoma cells were exhausted in hESCM4 culture after JARID1B knock down.

### Example 12

### JARID1B in Epithelial Carcinomas

Tissue samples from head and neck squamous cell carcinoma (HNSCC) were immunostained for JARID1B. JARID1B-positive cell clusters were found predominantly within the epithelial portion of the tumors. HNSCC cells with more mesenchymal phenotype (spindle-shaped cells) remained broadly negative. In this respect, a correlation between JARID1B and vimentin expression was not observed in the mesenchymal subpopulation of HNSCC in flow cytometry. This was of note because epithelial cells have been shown to gain sternness features after undergoing EMT (Mani, et al. (2008) supra). Taking into consideration that JARID1B is known to be highly expressed in breast cancer (Lu, et al. (1999) J. Biol. Chem. 274:15633-15645), there are several possible roles for JARID1B in carcinomas. For example, it could determine a population of slowly-proliferating epithelial cancer cells, which prepare to undergo EMT (and gain sternness). Alternatively, these epithelial clusters just represent local regions that are enriched for non-proliferating terminally differentiated cells. Upregulation also in differentiated carcinoma cells is generally conceivable since another jumonji/ARID family member, JARID2, is known to be strongly expressed in both embryonic stem cells and terminally differentiated cells whereas it is suppressed in proliferating tissue (Takeuchi, et al. (2006) Dev. Dyn. 235:2449-2459). Accordingly, strong JARID1B expression was observed in the basal layer of skin keratinocytes (the region where keratinocyte stem cells reside), a down-regulation of JARID1B in the highly proliferative second and third keratinocyte layers, and again an upregulation in terminally differentiated keratinocytes from the upper epidermis. Since label-retaining carcinoma cells, e.g., esophageal carcinoma, typically show a decreased colony formation capacity, it was assumed that the majority of slowly-cycling cells in epithelial carcinomas indeed represented rather differentiated cells which lost their potential to self-renew and initiate tumors. However, it is possible that JARID1B might be upregulated in slowly-proliferating carcinoma stem cells also.

### Example 13

### JARID1B+ Melanoma Cells are Less Susceptible to Conventional Cancer Therapy

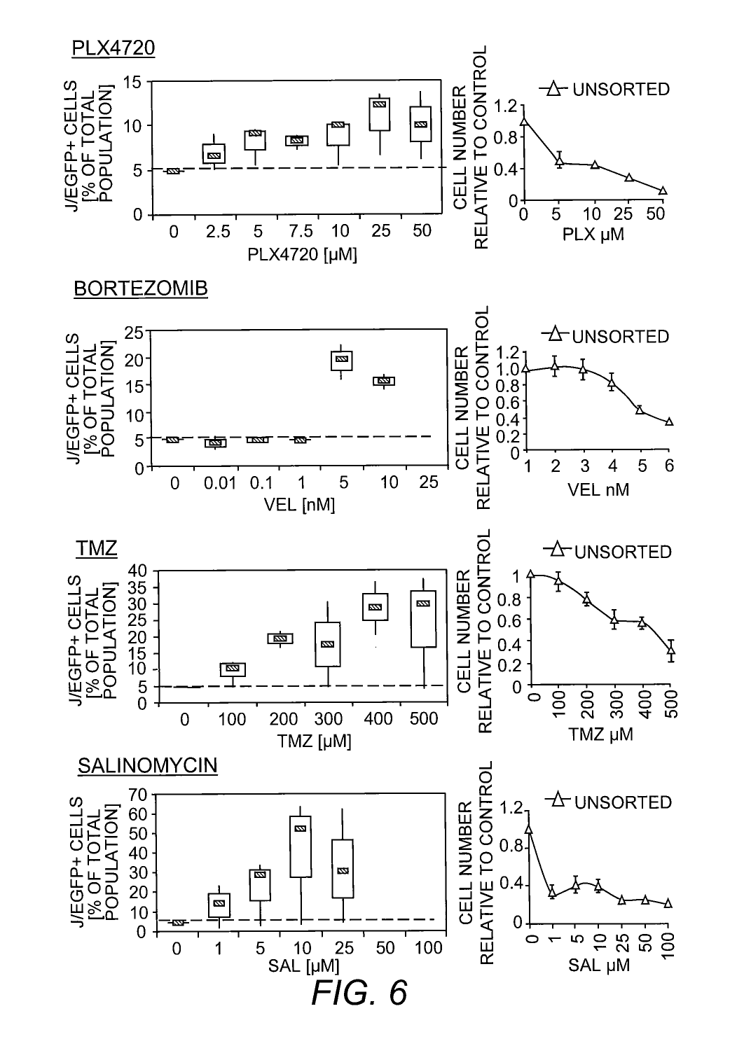

Treatment of WM3734^{JARID1Bprom-EGFP} cells with clinically relevant doses of a mutant-specific BRAFV600E inhibitor (PLX7420), temozolomide, bortezomib or salinomycin resulted in relative enrichment for the J/EGFP+ subpopulation (FIG. 6). Very low or very high drug concentrations did not have a discriminatory effect on the composition of the population. Since total numbers of viable cells were decreasing under drug treatment (FIG. 6, right panels) and primarily the J/EGFP− cells became positive for 7AAD, these data indicate that conventional drug treatment at therapeutic concentrations is selectively killing J/EGFP− cells, whereas J/EGFP+ cells survive. When J/EGFP+ and J/EGFP− cells were sorted by FACS prior to treatment, the differences in drug susceptibility were also visible but became less significant, highlighting the importance of a well-established niche for maintenance of the slow-cycling phenotype and its survival during therapy.

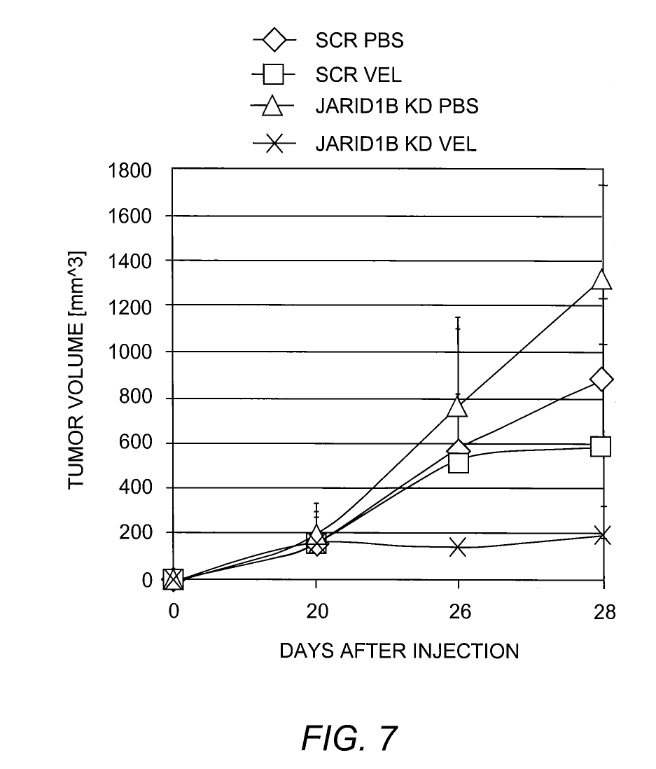

To further analyze the in vivo susceptibility of melanoma to conventional anti-cancer therapy in the presence of stable depletion of the JARID1B-expressing subpopulation, xenotransplanted WM3734 melanomas were treated with bortezomib after xenotransplantation. The results of this analysis indicated that JARID1B knocked-down tumors showed a significantly lower tumor volume (FIG. 7). This in vivo data indicate that when the JARID1B-positive subpopulation is depleted, secondary/conventional therapies can kill melanoma much more efficiently. Thus, a strategy to kill the entire tumor and prevent recurrences or therapy resistance requires eradicating the stem-like subpopulation of melanoma by targeting JARID1B and killing/debulking the rest of the tumor by, e.g., conventional approaches.

### Example 14

### Activation of JARID1B by Reconstitution

As an alternative approach to inhibition, JARID1B could also be activated in the treatment of cancer. Such activation can be achieved by reconstitution via transpermeable transduction of a TAT-JARID1B fusion protein. In this respect, the C-terminus of JARID1B (i.e., amino acid residues 1385-1582 of JARID1B, which is fully functional in terms of pRB stabilization), was fused in-frame with TAT (YGRKKRRQRRR; SEQ ID NO:16) via a Gly-Gly linker. The recombinant protein was contained a C-terminal 6×His-Tag and was produced in E. coli. Purified TAT-JARID1B was supplemented to culture media at 1 μM for 24 hours. TAT-fusion proteins were contacted with A375-SM melanoma cells and shown to stimulate cell death. Moreover, this effect could be enhanced by reducing serum content in the medium from 10% to 1%, which reduced competition for TAT binding sites.

1-71. (canceled)

72. A composition comprising a cancer therapeutic agent and a JARID1B inhibitor.

73. A pharmaceutical composition comprising the composition of claim 72 in admixture with a pharmaceutically acceptable carrier.

74. A method for decreasing self-renewal of tumor cells comprising contacting a tumor with a JARID1B inhibitor thereby decreasing self-renewal of the tumor cells.

75. A pharmaceutical composition comprising a JARID1B inhibitor in admixture with a pharmaceutically acceptable carrier, wherein said composition is formulated for transdermal or topical administration.

76. A method for treating cancer comprising administering to a subject in need of treatment an effective amount of a cancer therapeutic agent in combination with an agent that modulates JARID1B thereby treating the subject's cancer.

77. A method for inhibiting metastatic progression of a cancer comprising administering to a subject in need of treatment an effective amount of a JARID1B inhibitor thereby inhibiting metastatic progression of the subject's cancer.
